(19) United States
(12) Patent Application Publication
(10) Pub. No.: US 2024/0353418 A1
(43) Pub. Date: Oct. 24, 2024

(54) MASS SPECTROMETRY-CLEAVABLE HALOACETAMIDE-BASED CROSS-LINKERS AND USES THEREOF

(71) Applicant: The Regents of the University of California, Oakland, CA (US)

(72) Inventors: Lan Huang, Irvine, CA (US); Scott D. Rychnovsky, Irvine, CA (US)

(21) Appl. No.: 18/641,237

(22) Filed: Apr. 19, 2024

Related U.S. Application Data
(60) Provisional application No. 63/461,231, filed on Apr. 21, 2023.

(51) Int. Cl. G01N 33/68; G01N 33/58

(52) U.S. Cl. CPC G01N 33/6848; G01N 33/58; G01N 33/6842; G01N 33/6845

(57) ABSTRACT

The disclosure provides for mass spectrometry (MS)-cleavable haloacetamide cross-linkers, and uses thereof, including for proteome-wide analysis of protein-protein interactions.

### CROSS REFERENCE TO RELATED APPLICATIONS

This application claims priority under 35 U.S.C. § 119 from Provisional Application Ser. No. 63/461,231, filed Apr. 21, 2023 the disclosure of which is incorporated herein by reference.

### STATEMENT OF GOVERNMENT SUPPORT

This invention was made with Government support under Grant Nos. R01GM074830 and R01GM130144, awarded by the National Institutes of Health. The Government has certain rights in the invention

### INCORPORATION BY REFERENCE OF SEQUENCE LISTING

Accompanying this filing is a Sequence Listing entitled, “00058-078001.xml” created on Apr. 19, 2024 and having 4,613 bytes of data, machine formatted on IBM-PC, MS-Windows operating system. The sequence listing is hereby incorporated by reference in its entirety for all purposes.

### TECHNICAL FIELD

The disclosure provides for mass spectrometry (MS)-cleavable haloacetamide-based cross-linkers, and uses thereof, including for proteome-wide analysis of protein-protein interactions.

### BACKGROUND

Protein-protein interactions (PPIs) are essential for the assembly of protein complexes, the active molecular modules for controlling cellular functionality and modulating physiological states. In recent years, cross-linking mass spectrometry (XL-MS) has been proven effective for studying PPIs and elucidating architectures of protein complexes in vitro and in vivo at the systems-level. Compared to other PPI methods, XL-MS enables the capture of endogenous PPIs without cell engineering. Identification of cross-linked peptides concurrently reveals PPI identities and interaction contacts at specific residues, which provide distance constraints defined by a given cross-linker to help refine existing structures and elucidate structures of protein complexes through computational modeling. So far, only lysine-reactive cross-linkers have been successfully applied for proteome-wide PPI profiling. However, lysine cross-linkers alone cannot uncover the complete PPI map in cells.

### SUMMARY

The development of MS-cleavable cross-linkers have significantly advanced global PPI mapping to define the modularity and dynamics of cellular networks. This is due to the capability of MS-cleavable cross-linkers to provide simplified MS data for effective database searching, permitting cross-link identification with speed and accuracy. While successful, current proteome-wide XL-MS studies have all been relying on lysine-reactive cross-linkers. Although multiple cross-linking chemistries have been explored for XL-MS studies, lysine-reactive reagents remain the most popular. However, lysine-based XL-MS studies have only uncovered a fraction of proteomes. It is evident that additional cross-linking chemistries are important to expand PPI coverages and complement existing reagents. Provided herein are mass spectrometry (MS)-cleavable haloacetamide cross-linkers. While haloacetamides may have been shown to have slower cysteine reactivity compared with other cysteine interacting agents, they were found to be advantageous herein, in that they do not hydrolyze, are residue specific, and produce more homogenous cross-linked products for improved identification and quantitation. For the studies, a haloacetamide-based crosslinker was designed herein. This crosslinker, DBrASO (dibromoacetamide sulfoxide, aka, 3,3′-sulfinylbis(N′-(2-bromoacetyl) propanehydrazide) was generated by coupling bromoacetamide functional groups with sulfoxide-based MS-cleavability for unambiguous cross-link identification. It was demonstrated herein that DBrASO was an effective cysteine-reactive cross-linker for both simple and complex samples. Moreover, the disclosure provides for an innovative integrated DBrASO-based XL-MS analytical technique that couples a haloacetamide-based crosslinker of the disclosure with a newly developed two-dimensional fractionation method and LC-MSⁿ data acquisition for complex PPI profiling. This technique has been successfully applied to cross-link HEK293 cell lysates, yielding a total of 11,478 unique cross-linked peptides describing an XL-proteome containing 2,297 proteins. The studies presented herein represent the first proteome-wide XL-MS study using a cysteine cross-linker. The results have demonstrated that a haloacetamide-based crosslinker of the disclosure (e.g., DBrASO) is effective for global XL-MS analysis and represents an attractive reagent to complement existing lysine-reactive cross-linkers for comprehensive PPI mapping at the systems-level.

In a particular embodiment, the disclosure provides a mass spectrometry (MS)-cleavable haloacetamide-based cross-linker comprising: one or more terminal haloacetamide groups; a single, centrally located sulfoxide group; and one or more MS-cleavable bonds; wherein the MS-cleavable haloacetamide-based cross-linker is configured to specifically interact with cysteine groups for mapping intra-protein interactions in a protein, or inter-protein interactions in a protein complex, or combinations thereof. In a further embodiment, the one or more MS-cleavable bonds is/are C—S bond(s) adjacent to the single, centrally located sulfoxide group. In yet a further embodiment, the mass spectrometry (MS)-cleavable haloacetamide-based crosslinker has 2 terminal haloacetamide groups that are located equal distant to the single, centrally located sulfoxide group. In another embodiment, the haloacetamide group has a structure of:

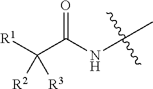

wherein, R¹-R³ are individually selected from H, D and halo, wherein at least one of R¹-R³ is a halo. In yet another embodiment, R¹ is Br; and R²-R³ are H. In a further embodiment, the MS-cleavable haloacetamide-based cross-linker further comprises a first linker group, wherein one end of the first linker arm is connected to the single, centrally located sulfoxide group and the other end of the first linker group is connected to one of the terminal haloacetamide groups. In yet a further embodiment, the MS-cleavable haloacetamide-based cross-linker further comprises a second linker group, wherein one end of the second linker group is connected to the central sulfoxide group and the other end of the second linker group is connected to another terminal haloacetamide group, and wherein the first linker group and the second linker groups comprise identical functional groups and are symmetric. In a certain embodiment, the MS-cleavable haloacetamide-based cross-linker is DBrASO having the structure of:

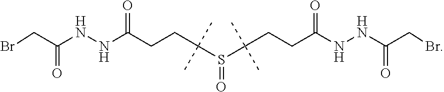

In a particular embodiment, the disclosure also provides a mass spectrometry (MS)-cleavable haloacetamide-based cross-linker having the structure of Formula (I):

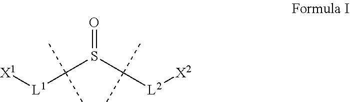

wherein, X¹ and X² are independently selected haloacetamide groups; L¹ and L² are independently selected linker groups; and wherein the dashed line indicates a MS-cleavable bond. In a further embodiment, X¹ and X² comprises the structure of

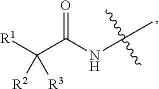

and wherein, R¹-R³ are individually selected from H, D or halo, wherein at least one of R¹-R³ is a halo. In yet a further embodiment, R¹ is Br; and R²-R³ are H. In another embodiment, L¹ and L² are individually selected from an optionally substituted (C₁-C₁₂)alkyl, an optionally substituted (C₁-C₁₂)alkenyl,

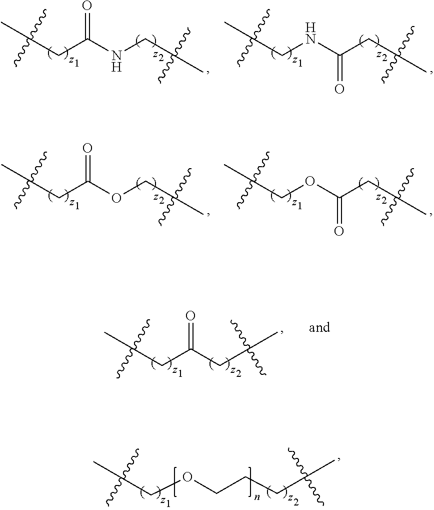

wherein, z is an integer selected from 0, 1, 2, 3, 4, 5, 6, 7, and 8; z² is an integer selected from 1, 2, 3, 4, 5, 6, 7, and 8; and n is an integer selected from 1, 2, 3, and 4. In yet another embodiment, L¹ and L² are selected from,

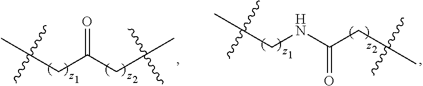

wherein, z¹ is an integer selected from 0, 1, 2, 3, and 4; and z² is an integer selected from 1, 2, 3, and 4. In a further embodiment, Lt and L² are

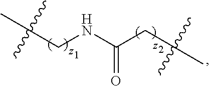

wherein, z¹ is an integer selected from 0, 1, 2 and 3; and z² is an integer selected from 2, 3, and 4. In a certain embodiment, L¹ and L² are selected from

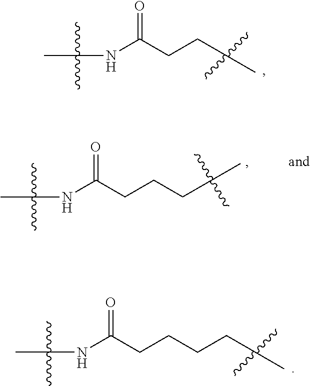 [SMILES: *CCC(=O)N* ; *CCCC(=O)N* ; *CCCCC(=O)N*]

In a further embodiment, the MS-cleavable haloacetamide-based cross-linker has a structure of:

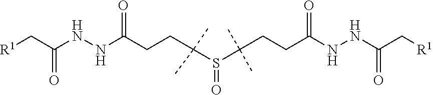

wherein, R¹ is a halo selected from Cl, Br, and I. In a certain embodiment, the MS-cleavable haloacetamide-based cross-linker is DBrASO having the structure of:

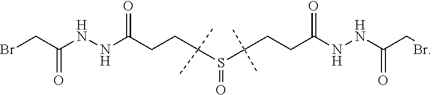

In a particular embodiment, the disclosure also provides a method for mapping intra-protein interactions in a protein, inter-protein interactions in a protein complex, or any combination thereof, the method comprising: contacting the protein and/or the protein complex comprising a plurality of cysteine moieties with the MS-cleavable haloacetamide-based cross-linker of any one of aspects 1 to 23 to form a cross-linked product; and digesting the cross-linked product to form a plurality of fragments, wherein a portion of the plurality of fragments comprises cross-linked peptide fragments; identifying and analyzing cross-linked peptide fragments using tandem mass spectrometry (MSⁿ) to map intra-protein interactions in the protein and/or inter-protein interactions in the protein complex. In a further embodiment, a data-dependent MS³ acquisition method is used for identifying and analyzing the cross-linked peptide fragments.

In a certain embodiment, the disclosure further provides a method for mapping global protein-protein interactions (PPIs) from a sample comprising a plurality of proteins; contacting the sample comprising a plurality of proteins with the MS-cleavable haloacetamide-based cross-linker of claim 1 to form crosslinked proteins; digesting the crosslinked proteins to form crosslinked protein fragments or peptides; isolating fractions that are enriched with cross-linked protein fragments or peptides in the sample; analyzing the fractions using tandem mass spectrometry (MSⁿ) and protein database searching to identify cross-linked protein fragments or peptides; and mapping the identified cross-linked protein fragments or peptides to generate a global structural map of PPIs. In a further embodiment, the fractions are isolated by using peptide size exclusion chromatography coupled with high pH reverse phase tip fractionation.

In a certain embodiment, the disclosure provides a composition, a method or a kit as substantially described herein.

### DESCRIPTION OF DRAWINGS

FIG. 1 presents an exemplary embodiment of a synthesis pathway for DBrASO.

FIG. 2A-C presents schematics of the sulfoxide-containing MS-cleavable cysteine-reactive cross-linkers. Structures of (A) BMSO and (B) DBrASO. (C) Predicted MS² fragmentation of a DBrASO interlinked peptide.

FIG. 3A-D provides MSⁿ analysis of the DBrASO inter-linked Ac-LR9 peptide (SEQ ID NO:1). (A) MS¹ spectrum of the inter-linked Ac-LR9, (α-α)⁴⁺,(m/z 603.7698⁴⁺). (B) MS² spectrum of the (α-α)⁴⁺ detected by MS¹, containing 3 predicted fragments, α_{A} (m/z 591.2739²⁺), α_{T} (m/z 607.2593²⁺) and α_{S} (m/z 616.2654²⁺). (C) MS³ analysis of α_{T} (m/z 607.2593²⁺). (D) MS³ analysis of α_{A} (m/z 591.2739²⁺).

FIG. 4A-D presents MSⁿ analysis of a representative DBrASO interlinked peptide of BSA. (A) DBrASO interlinked peptide was detected as a quadruple charged ion (α-β) (m/z 676.5522⁴⁺) in MS¹. (B) MS² spectrum of the cross-linked peptide (α-β) in A, in which two expected fragment ion pairs were detected, α_{A}/β_{T} (m/z 571.2717²⁺/772.8250²⁺) and α_{T}/β_{A} (m/z 587.2579²⁺/756.8391²⁺). (C) MS³ analysis of α_{A}(m/z 571.2717²⁺) identified the sequence as SHCAIAEVEK (SEQ ID NO:2), in which the cysteine residue was modified with alkene (A). (D) MS³ analysis of β_{T} (m/z 772.8250²⁺) identified the sequence as YICTDNQDTISSK (SEQ ID NO:3), in which the cysteine residue was modified with thiol (T).

FIG. 5A-C provides C-C XL-maps of BSA (PDB: 4F5S). Three-dimensional (3D) XL-map from (A) DBrASO and (B) BMSO crosslinked data. (C) Distance distribution plots of DBrASO and BMSO C-C linkages.

FIG. 6 shows the overlap of DBrASO and BMSO cross-linked C-C linkages of BSA.

FIG. 7 presents the general in vitro XL-MS workflow of cell lysates using DBrASO.

FIG. 8A-C provides a comparison of DBrASO and DSSO XL-Proteomes of HEK293 cell lysates. (A) Subcellular compartment distribution of human proteome (from UniProt) and DBrASO cross-linked proteins. (B) Overlap of DSSO and DBrASO cross-linked proteins. (C) iBAQbased abundance distribution of DBrASO (red) and DSSO (blue) XL-proteomes and MS-proteome from Proteomics DB (gray).

FIG. 9A-F displays gene ontology analysis of DBrASO and DSSO cross-linked proteins from HEK293 cell lysates. (A) cellular component, (B) biological process and (C) molecular function analysis of DBrASO cross-linked proteins. (D) cellular component, (E) biological process and (F) molecular function analysis of DSSO cross-linked proteins.

FIG. 10 provides iBAQ-based abundance distribution of DBrASO cross-linked proteins from HEK293 cell lysates and their proportion of cysteines.

FIG. 11A-D shows the characterization of C-C cross-links by structural mapping. Mapping DBrASO cross-links onto high-resolution 3D structures of (A) the CCT complex (PDB: 7LUP), (B) 26S proteasome (PDB:5GJR), and (C) pre-B spliceosome complex (PDB: 6QX9). Satisfied cross-links (≤40 Å) were labeled blue and nonsatisfied cross-linked (>40 Å) were labeled red. (D) Distance distributions of the identified cross-links from the three selected complexes.

FIG. 12A-B provides a comparison of DBrASO and DSSO XL-PPIs. (A) XL-PPI network of DBrASO cross-linked HEK 293 cell lysates. The DBrASO XL-PPI network is derived from 1,148 inter-protein C-C linkages, composed of 505 node and 856 edges. Blue edge: PPI identified by both DSSO and DBrASO, red edge: PPI identified by DBrASO only. (B) Overlap of PPIs identified from DSSO and DBrASO cross-linked HEK 293 cell lysates.

FIG. 13 presents STRING score distributions for PPIs in STRING database (red) and XL-PPIs (blue) determined in this work.

FIG. 14 demonstrates GAPDH-containing XL-PPI networks by DBrASO cross-linking. GAPDH-interacting proteins were grouped by protein families. The reported PPIs were labeled by grey edges, and the novel PPIs were labeled by green edges.

FIG. 15 presents an overview of process for global mapping of protein-protein interactions using DBrASO, a haloacetamide-based cysteine crosslinker.

### DETAILED DESCRIPTION

As used herein and in the appended claims, the singular forms “a,” “an,” and “the” include plural referents unless the context clearly dictates otherwise. Thus, for example, reference to “a crosslinker” includes a plurality of such crosslinkers and reference to “the sulfoxide group” includes reference to one or more sulfoxide groups and equivalents thereof known to those skilled in the art, and so forth.

It is to be further understood that where descriptions of various embodiments use the term “comprising,” those skilled in the art would understand that in some specific instances, an embodiment can be alternatively described using language “consisting essentially of” or “consisting of.”

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “alkenyl”, as used in this disclosure, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, as used in this disclosure, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, as used in this disclosure, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompasses from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “functional group” or “FG” refers to specific groups of atoms within molecules that are responsible for the characteristic chemical reactions of those molecules. While the same functional group will undergo the same or similar chemical reaction(s) regardless of the size of the molecule it is a part of, its relative reactivity can be modified by nearby functional groups. The atoms of functional groups are linked to each other and to the rest of the molecule by covalent bonds. Examples of FGs that can be used in this disclosure, include, but are not limited to, halogens, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, sulfhydryls, sulfides, disulfides, sulfinyls, sulfos, thiocyanates, isothiocyanates, carbonothioyls, phosphinos, phosphonos, and phosphates.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

The term “substituent” refers to an atom or group of atoms substituted in place of a hydrogen atom. For purposes of this invention, a substituent would include deuterium atoms. Examples of substituents that can replace a hydrogen group in the structure of a crosslinker disclosed herein include, but are not limited to, halogen, hydroxyl, carboxyl, aldehyde, nitrile, isonitrile, nitro, amino, sulfide, alkyl (e.g., (C₁-C₆)alkyl), alkenyl (e.g., (C₁-C₆)alkenyls), alkynyl (e.g., (C₁-C₆)alkynyl), alkoxy (e.g., (C₁-C₆) alkoxy, ester (e.g., (C₁-C₆) ester), aryl, cycloalkyl, and heterocycle.

The term “substituted” with respect to hydrocarbons, heterocycles, and the like, refers to structures wherein the parent chain contains one or more substituents.

The term “unsubstituted” with respect to hydrocarbons, heterocycles, and the like, refers to structures wherein the parent chain contains no substituents.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Protein-protein interactions (PPIs) are essential for the assembly of protein complexes, the active molecular modules for controlling cellular functionality and modulating physiological states. In recent years, cross-linking mass spectrometry (XL-MS) has been proven effective for studying PPIs and elucidating architectures of protein complexes in vitro and in vivo at the systems-level. Compared to other PPI methods, XL-MS enables the capture of endogenous PPIs without cell engineering. Identification of cross-linked peptides concurrently reveals PPI identities and interaction contacts at specific residues, which provide distance constraints defined by a given cross-linker to help refine existing structures and elucidate structures of protein complexes through computational modeling. In particular, the development of MS-cleavable cross-linkers has significantly advanced global PPI mapping to define the modularity and dynamics of cellular networks. This is due to the capability of MS-cleavable cross-linkers to provide simplified MS data for effective database searching, permitting cross-link identification with speed and accuracy. While successful, current proteome-wide XL-MS studies have all been relying on lysine-reactive cross-linkers. Although multiple cross-linking chemistries have been explored for XLMS studies lysine-reactive reagents remain the most popular. However, lysine-based XL-MS studies have only uncovered a fraction of proteomes. It is evident that additional cross-linking chemistries are important to expand PPI coverages and complement existing reagents. In addition to lysine and acidic residues, cysteine is an attractive and unique amino acid owing to its critical importance in protein structure and function. Since the sulfhydryl group is highly reactive and can form disulfide bonds, cysteines modulate protein structures in multiple way including protein dimerization, metal coordination, redox regulation, and thermal stability. Cysteines can be labeled by many electrophilic reagents with high specificity and efficiency, which has been widely used in proteomic studies. While cysteine is one of the least abundant amino acids, it is often found at functional sites of proteins and its evolution appears to be highly regulated in proteins. Thus, cysteine cross-linking is expected to provide additional molecular details to help advance the understanding of protein interactions and structures. The homobifunctional MS-cleavable cysteine-reactive cross-linker bismaleimide sulfoxide (BMSO) has been used to define protein interactions and can be used in connection with lysine and acidic residue reactive reagents to expand PPI coverages. In addition, the multi-chemistry-based XL-MS approach was shown to facilitate the elucidation of the architectures of protein complexes by improving structural modeling with significantly increased precision. While maleimide chemistry has the fastest kinetics among the commonly used cysteine reactive groups, maleimide-labeled products can undergo retro-Michael addition and thiol exchange under physiological conditions. In addition, maleimides are prone to hydrolysis, which leads to the formation of ring-opened maleamic acids that are inactive to thiols. Finally, maleimide chemistry can be promiscuous, capable of labeling lysines at alkaline pH and resulting in unexpected complexity. Therefore, the exploration of alternative cysteine-labeling chemistries with higher specificity and less complexity would be advantageous to enable robust profiling of cysteine XL-proteomes for expansion of PPI mapping.

Provided herein are mass spectrometry (MS)-cleavable haloacetamide cross-linkers. While haloacetamides may have been shown to have slower cysteine reactivity compared with other cysteine interacting agents, they were found to be advantageous herein, in that they do not hydrolyze, are residue specific, and produce more homogenous cross-linked products for improved identification and quantitation. For the studies, a haloacetamide-based crosslinker was designed herein. This crosslinker, DBrASO (dibromoacetamide sulfoxide, aka, 3,3′-sulfinylbis(N′-(2-bromoacetyl) propanehydrazide) was generated by coupling bromoacetamide functional groups with sulfoxide-based MS-cleavability for unambiguous cross-link identification. It was demonstrated herein that DBrASO was an effective cysteine-reactive cross-linker for both simple and complex samples. Moreover, the disclosure provides for an innovative integrated DBrASO-based XL-MS analytical technique that couples a haloacetamide-based crosslinker of the disclosure with a newly developed two-dimensional fractionation method and LC-MSⁿ data acquisition for complex PPI profiling. This technique has been successfully applied to cross-link HEK293 cell lysates, yielding a total of 11,478 unique cross-linked peptides describing an XL-proteome containing 2,297 proteins. The studies presented herein represent the first proteome-wide XL-MS study using a cysteine cross-linker. The results have demonstrated that a haloacetamide-based crosslinker of the disclosure (e.g., DBrASO) is effective for global XL-MS analysis and represents an attractive reagent to complement existing lysine-reactive cross-linkers for comprehensive PPI mapping at the systems-level.

In a particular embodiment, the disclosure provides a mass spectrometry (MS)-cleavable haloacetamide-based cross-linker comprising: one or more terminal haloacetamide groups; a single, centrally located sulfoxide group; and one or more MS-cleavable bonds; wherein the MS-cleavable haloacetamide-based cross-linker is configured to specifically interact with cysteine groups for mapping intra-protein interactions in a protein, or inter-protein interactions in a protein complex, or combinations thereof. In a further embodiment, the one or more MS-cleavable bonds is/are C—S bond(s) adjacent to the single, centrally located sulfoxide group. In another embodiment, fragmentation of the MS-cleavable haloacetamide-based cross-linker by mass spectrometry produces two major ion pairs. In yet another embodiment, the MS-cleavable haloacetamide-based cross-linker has little to no reactivity for lysine moieties. In a further embodiment, the MS-cleavable haloacetamide-based cross-linker has 1, 2, 3, 4, 5, 6, 7 or 8, or a range that includes, or is in between any two of the foregoing numbers, terminal haloacetamide groups. In a particular embodiment, the MS-cleavable haloacetamide-based cross-linker has 2 or 3 terminal haloacetamide groups. In a further embodiment, the MS-cleavable haloacetamide-based cross-linker has 2 terminal haloacetamide groups. In another embodiment, the mass spectrometry (MS)-cleavable haloacetamide-based crosslinker has 2 terminal haloacetamide groups that are located equal distant to the single, centrally located sulfoxide group. In yet another embodiment, the one or more terminal haloacetamide groups have a structure of:

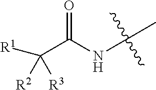

wherein, R¹-R³ are individually selected from H, D and halo, wherein at least one of R¹-R³ is a halo. In a further embodiment, R¹ is a halo group selected from I, Br, and Cl; and R²-R³ are H. In yet a further embodiment, R¹ is Br. In a certain embodiment, the haloacetamide-based cross-linker is a homobifunctional cross-linker. In another embodiment, the mass spectrometry (MS)-cleavable haloacetamide-based cross-linker further comprises a first linker arm, wherein one end of the first linker group is connected to the single, centrally located sulfoxide group and the other end of the first linker group is connected to one of the terminal haloacetamide groups. In yet another embodiment, the mass spectrometry (MS)-cleavable haloacetamide-based cross-linker further comprises a second linker group, wherein one end of the second linker arm is connected to the single, centrally located sulfoxide group and the other end of the second linker arm is connected to another terminal haloacetamide group, and wherein the first linker group and the second group arm comprise identical groups and are symmetric. In yet another embodiment, the MS-cleavable haloacetamide-based cross-linker is DBrASO having the structure of:

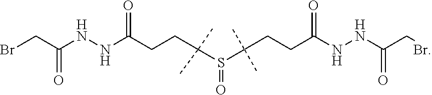

In a particular embodiment, the disclosure provides a mass spectrometry (MS)-cleavable haloacetamide-based cross-linker having the structure of Formula (I):

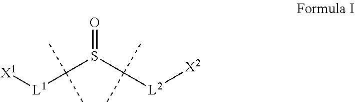

wherein,
X¹ and X² are independently selected haloacetamide groups;
L¹ and L² are independently selected linker groups; and wherein the dashed line indicates a MS-cleavable bond. In a further embodiment, X¹ and X² comprises the structure of

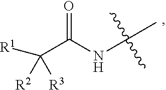

and wherein, R¹-R³ are individually selected from H, D and halo, and wherein at least one of R¹-R³ is a halo. In yet a further embodiment, R¹ is Br; and R²-R³ are H. In another embodiment, L¹ and L² are individually selected from an optionally substituted (C₁-C₁₂)alkyl, an optionally substituted (C₁-C₁₂)alkenyl,

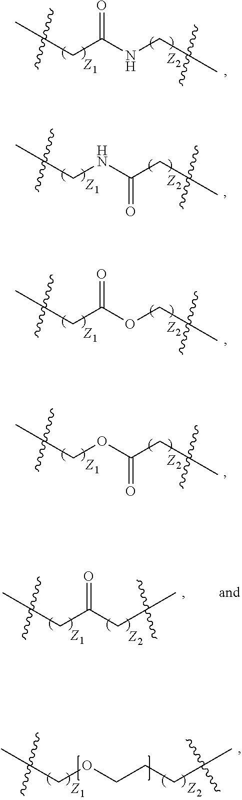

wherein, z¹ is an integer selected from 0, 1, 2, 3, 4, 5, 6, 7, and 8; z² is an integer selected from 1, 2, 3, 4, 5, 6, 7, and 8; and n is an integer selected from 1, 2, 3, and 4. In a further embodiment, L¹ and L² are selected from

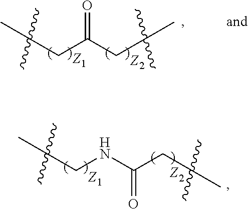

wherein, z¹ is an integer selected from 0, 1, 2, 3, and 4; and z² is an integer selected from 1, 2, 3, and 4. In yet a further embodiment, L¹ and L² are

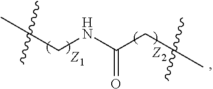

wherein, z is an integer selected from 0, 1, 2 and 3; and z² is an integer selected from 2, 3, and 4. In another embodiment, L¹ and L² are selected from

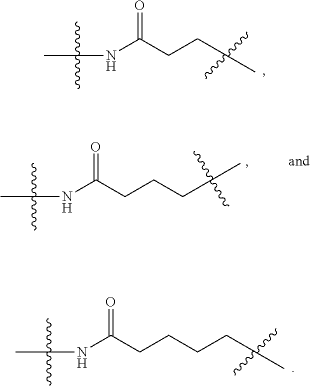 [SMILES: *CCC(=O)N* ; *CCCC(=O)N* ; *CCCCC(=O)N*]

In yet another embodiment, L¹ and L² have a length of about 10 Å, about 11 Å, about 12 Å, about 13 Å, about 14 Å, about 15 Å, about 16 Å, about 17 Å, about 18 Å, about 19 Å, or about 20 Å, or a range that includes or is between any two of the foregoing distances (e.g., from 15 Å to 20 Å). In another embodiment, the MS-cleavable haloacetamide-based cross-linker has a structure of:

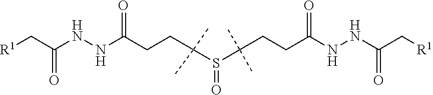

wherein, R¹ is a halo selected from Cl, Br, and I. In yet another embodiment, the MS-cleavable haloacetamide-based cross-linker is DBrASO having the structure of:

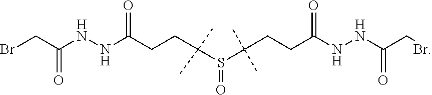

The disclosure also provides methods or processes for mapping intra-protein interactions in a protein, inter-protein interactions in a protein complex, or any combination thereof. In a particular embodiment, the method comprises: contacting the protein and/or the protein complex comprising a plurality of cysteine moieties with the MS-cleavable haloacetamide-based cross-linker of any one of aspects 1 to 23 to form a cross-linked product; digesting the cross-linked product to form a plurality of fragments, wherein a portion of the plurality of fragments comprises cross-linked peptide fragments; and identifying and analyzing cross-linked peptide fragments using tandem mass spectrometry (MSⁿ) to map intra-protein interactions in the protein and/or inter-protein interactions in the protein complex. In a further embodiment, the MS-cleavable haloacetamide-based cross-linker is used at a molar ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1, or a range that includes or is between any two of the foregoing ratios (e.g., 1:5 to 5:1), to the protein and/or the protein complex. In a further embodiment, the MS-cleavable haloacetamide-based cross-linker is used at a molar ratio of 1:5 to 5:1 to the protein and/or the protein complex. In yet a further embodiment, the cross-linked product was digested with one or more proteases. Examples of proteases include, but are not limited to, serine proteases, threonine proteases, aspartic proteases, glutamic proteases, metalloproteases, and asparagine peptide lyases. In a particular embodiment, the one or more proteases are serine proteases. In another embodiment, a data-dependent MS³ acquisition method is used for identifying and analyzing the cross-linked peptide fragments.

The disclosure also provides methods for mapping global protein-protein interactions (PPIs) from a sample comprising a plurality of proteins using a MS-cleavable haloacetamide-based cross-linker disclosed herein. In a particular embodiment, a method for mapping global protein-protein interactions (PPIs) from a sample comprising a plurality of proteins comprises: contacting the sample comprising a plurality of proteins with the MS-cleavable haloacetamide-based cross-linker of claim 1 to form crosslinked proteins; digesting the crosslinked proteins to form crosslinked protein fragments or peptides; isolating fractions that are enriched with cross-linked protein fragments or peptides in the sample; analyzing the fractions using tandem mass spectrometry (MSn) and protein database searching to identify cross-linked protein fragments or peptides; and mapping the identified cross-linked protein fragments or peptides to generate a global structural map of PPIs. In a further embodiment, the sample is a cellular sample. In yet a further embodiment, the cross-linked product was digested with one or more proteases. Examples of proteases include, but are not limited to, serine proteases, threonine proteases, aspartic proteases, glutamic proteases, metalloproteases, and asparagine peptide lyases. In another embodiment, the one or more proteases are serine proteases. In yet another embodiment, the fractions are isolated by using peptide size exclusion chromatography coupled with high pH reverse phase tip fractionation. In a further embodiment, a data-dependent MS³ acquisition method is used for identifying the cross-linked protein fragments or peptides. In yet a further embodiment, the identified cross-linked protein fragments or peptide are mapped using various databases that profile protein to protein interactions. Examples of databases that profile protein to protein interactions, include, but are not limited to, APID, MiMI, iRefIndex, String, BioGrid, HPIDB, MINT, DIP, IntAct, HPRD, CORUM, and BioPlex. In yet another embodiment, the methods for mapping global protein-protein interactions (PPIs) further comprises the use of one or more additional MS-cleavable cross-linkers (e.g., BMSO, DSSO, DHSO, SDASO-S, Azide-A-DSBSO, Alkyne-A-DSBSO) with a MS-cleavable haloacetamide-based cross-linker disclosed herein.

Kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more MS-cleavable haloacetamide cross-linkers disclosed herein, optionally in a composition or in combination with another agent as disclosed herein. The container(s) typically are made to exclude or limit light exposure for the contents of the container. Such kits optionally comprise a MS-cleavable haloacetamide cross-linker disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a MS-cleavable haloacetamide-based cross-linker described herein. Non-limiting examples of such materials include, but are not limited to, buffers, diluents; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific analytical application. The label can also indicate directions for use of the contents, such as in the methods described herein.

The disclosure further provides that the methods and compositions described herein can be further defined by the following aspects (aspects 1 to 36):

1. A mass spectrometry (MS)-cleavable haloacetamide-based cross-linker comprising:
one or more terminal haloacetamide groups;
a single, centrally located sulfoxide group; and
one or more MS-cleavable bonds;
wherein the MS-cleavable haloacetamide-based cross-linker is configured to specifically interact with cysteine groups for mapping intra-protein interactions in a protein, or inter-protein interactions in a protein complex, or combinations thereof.

2. The MS-cleavable haloacetamide-based cross-linker of any one of aspect 1, wherein the one or more MS-cleavable bonds is/are C—S bond(s) adjacent to the single, centrally located sulfoxide group.

3. The MS-cleavable haloacetamide-based cross-linker of aspect 1 or aspect 2, wherein fragmentation of the MS-cleavable haloacetamide-based cross-linker by mass spectrometry produces two major ion pairs.

4. The MS-cleavable haloacetamide-based cross-linker of any one of aspects 1 to 3, wherein the MS-cleavable haloacetamide-based cross-linker has little to no reactivity for lysine moieties.

5. The MS-cleavable haloacetamide-based cross-linker of any one of aspects 1 to 4, wherein the MS-cleavable haloacetamide-based cross-linker has 1, 2, 3 or 4 terminal haloacetamide groups.

6. The MS-cleavable haloacetamide-based cross-linker of any one of aspects 1 to 5, wherein the MS-cleavable haloacetamide-based cross-linker has 2 or 3 terminal haloacetamide groups.

7. The MS-cleavable haloacetamide-based cross-linker of any one of aspects 1 to 6, wherein the MS-cleavable haloacetamide-based cross-linker has 2 terminal haloacetamide groups.

8. The MS-cleavable haloacetamide-based cross-linker of any one of aspects 1 to 7, wherein the mass spectrometry (MS)-cleavable haloacetamide-based crosslinker has 2 terminal haloacetamide groups that are located equal distant to the single, centrally located sulfoxide group.

9. The MS-cleavable haloacetamide-based cross-linker of any one of aspects 1 to 8, wherein the one or more terminal haloacetamide groups have a structure of:

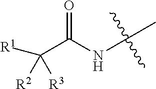

wherein, R¹-R³ are individually selected from H, D and halo, wherein at least one of R¹-R³ is a halo.

10. The MS-cleavable haloacetamide-based cross-linker of aspect 9, wherein R¹ is a halo group selected from I, Br, and Cl; and R²-R³ are H.

11. The MS-cleavable haloacetamide-based cross-linker of aspect 10, wherein R¹ is Br.

12. The MS-cleavable haloacetamide-based cross-linker of any one of aspects 1 to 11, wherein the haloacetamide-based cross-linker is a homobifunctional cross-linker.

13. The MS-cleavable haloacetamide-based cross-linker of any one of aspects 1 to 12, wherein the mass spectrometry (MS)-cleavable haloacetamide-based cross-linker further comprises a first linker group, wherein one end of the first linker arm is connected to the single, centrally located sulfoxide group and the other end of the first linker group is connected to one of the terminal haloacetamide groups.

14. The MS-cleavable haloacetamide-based cross-linker of any one of aspects 1 to 13, wherein the mass spectrometry (MS)-cleavable haloacetamide-based cross-linker further comprises a second linker group, wherein one end of the second linker arm is connected to the single, centrally located sulfoxide group and the other end of the second linker group is connected to another terminal haloacetamide group, and wherein the first linker arm and the second linker arm comprise identical groups and are symmetric.

15. A mass spectrometry (MS)-cleavable haloacetamide-based cross-linker having the structure of Formula (I):

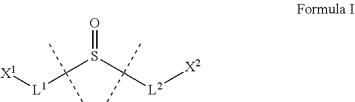

wherein,
X¹ and X² are independently selected haloacetamide groups;
L¹ and L² are independently selected linker groups; and wherein the dashed line indicates a MS-cleavable bond.

16. The MS-cleavable haloacetamide-based cross-linker of aspect 15,
wherein X¹ and X² comprises the structure of

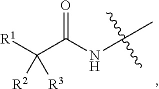

and wherein, R¹-R³ are individually selected from H, D and halo, and wherein at least one of R¹-R³ is a halo.

17. The MS-cleavable haloacetamide-based cross-linker of aspect 16, wherein R¹ is Br; and R²-R³ are H.

18. The MS-cleavable haloacetamide-based cross-linker of any one of aspects 15 to 17, wherein L¹ and L² are individually selected from an optionally substituted (C₁-C₁₂)alkyl, an optionally substituted (C₁-C₁₂)alkenyl,

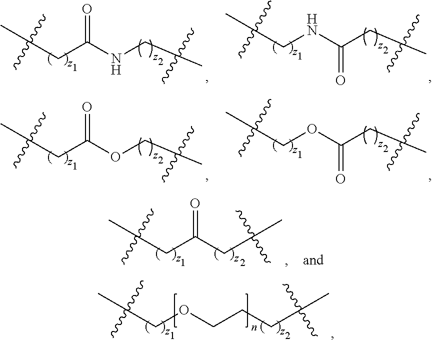

wherein, z¹ is an integer selected from 0, 1, 2, 3, 4, 5, 6, 7, and 8; z² is an integer selected from 1, 2, 3, 4, 5, 6, 7, and 8; and n is an integer selected from 1, 2, 3, and 4.

19. The MS-cleavable haloacetamide-based cross-linker of any one of aspects 15 to 18, wherein L¹ and L² are selected from

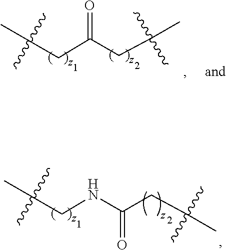

wherein, z¹ is an integer selected from 0, 1, 2, 3, and 4; and z² is an integer selected from 1, 2, 3, and 4.

20. The MS-cleavable haloacetamide-based cross-linker of any one of aspects 15 to 19, wherein L¹ and L² are

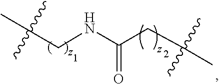

wherein, z¹ is an integer selected from 0, 1, 2 and 3; and z² is an integer selected from 2, 3, and 4.

21. The MS-cleavable haloacetamide-based cross-linker of any one of aspects 15 to 20, wherein L¹ and L² are selected from

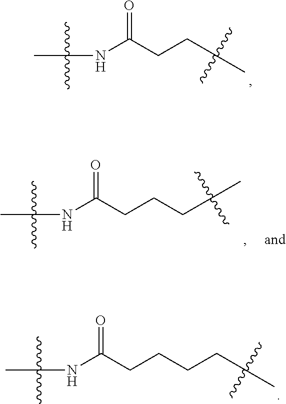 [SMILES: *CCC(=O)N* ; *CCCC(=O)N* ; *CCCCC(=O)N*]

22. The MS-cleavable haloacetamide-based cross-linker any one of aspects 15 to 21, wherein L¹ and L² have a length less than 20 Å.

23. The MS-cleavable haloacetamide-based cross-linker of any one of aspects 1 to 22, wherein the MS-cleavable haloacetamide-based cross-linker has a structure of:

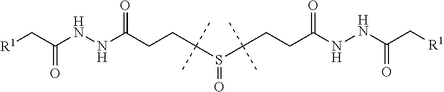

wherein, R¹ is a halo selected from Cl, Br, and I.

24. The MS-cleavable haloacetamide-based cross-linker of any one of aspects 1 to 23, wherein the MS-cleavable haloacetamide-based cross-linker is DBrASO having the structure of:

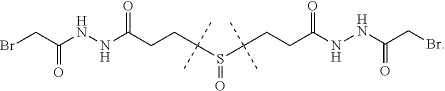

25. A method for mapping intra-protein interactions in a protein, inter-protein interactions in a protein complex, or any combination thereof, the method comprising:
contacting the protein and/or the protein complex comprising a plurality of cysteine moieties with the MS-cleavable haloacetamide-based cross-linker of any one of aspects 1 to 23 to form a cross-linked product;
digesting the cross-linked product to form a plurality of fragments, wherein a portion of the plurality of fragments comprises cross-linked peptide fragments; and
identifying and analyzing cross-linked peptide fragments using tandem mass spectrometry (MSⁿ) to map intra-protein interactions in the protein and/or inter-protein interactions in the protein complex.

26. The method of aspect 25, wherein the MS-cleavable haloacetamide-based cross-linker is used at a molar ratio of 1:10 to 10:1 to the protein and/or the protein complex.

27. The method of aspect 26, wherein the MS-cleavable haloacetamide-based cross-linker is used at a molar ratio of 1:5 to 5:1 to the protein and/or the protein complex.

28. The method of any one of aspects 24 to 27, wherein the cross-linked product was digested with one or more proteases.

29. The method of aspect 28, wherein the one or more proteases are serine proteases.

30. The method of any one of aspects 24 to 29, wherein a data-dependent MS³ acquisition method is used for identifying and analyzing the cross-linked peptide fragments.

31. A method for mapping global protein-protein interactions (PPIs) from a sample comprising a plurality of proteins;
contacting the sample comprising a plurality of proteins with the MS-cleavable haloacetamide-based cross-linker of claim 1 to form crosslinked proteins;
digesting the crosslinked proteins to form crosslinked protein fragments or peptides;
isolating fractions that are enriched with cross-linked protein fragments or peptides in the sample;
analyzing the fractions using tandem mass spectrometry (MSⁿ) and protein database searching to identify cross-linked protein fragments or peptides; and
mapping the identified cross-linked protein fragments or peptides to generate a global structural map of PPIs.

32. The method of aspect 31, wherein the sample is a cellular sample.

33. The method of any one of aspect 31 or aspect 32, wherein the cross-linked product was digested with one or more proteases.

34. The method of aspect 33, wherein the one or more proteases are serine proteases.

35. The method of any one of aspects 31 to 34, wherein the fractions are isolated by using peptide size exclusion chromatography coupled with high pH reverse phase tip fractionation.

36. The method of any one of aspects 34 to 35, wherein a data-dependent MS³ acquisition method is used for identifying the cross-linked protein fragments or peptides.

37. The method of any one of aspects 30 to 36, wherein the identified cross-linked protein fragments or peptide are mapped using various databases that profile protein to protein interactions.

The following examples are intended to illustrate but not limit the disclosure. While they are typical of those that might be used, other procedures known to those skilled in the art may alternatively be used.

### EXAMPLES

Materials and Reagents. General chemicals were purchased from Fisher Scientific or VWR International. Bovine serum albumin (BSA) (≥96% purity) was purchased from Sigma-Aldrich. Sequencing-grade trypsin was purchased from Promega (Madison, WI). Ac-LR9 peptide (Ac-LADVCAHER (SEQ ID NO:1, 98% purity) was custom ordered from Biomatik (Wilmington, DE).

Synthesis and Characterization of DBrASO and Relevant Intermediates. The known bis-ester 2-S1 was treated with t-butyl carbazate to furnish the bis-protected hydrazide 2-S2. The protecting groups were removed upon treatment with TFA and the resulting salt was stirred with basic resin Amberlyst A21 to afford the free hydrazide 2-S3. To form the bromoacetamides, the bis-hydrazide was reacted with ester 2-S4 according to Trmčič et al. (Beilstein journal of organic chemistry 6:732-741 (2010)). The resulting bromoacetamide 2-S5 was finally oxidized from the sulfide to the sulfoxide to afford the cross-linker, DBrASO.

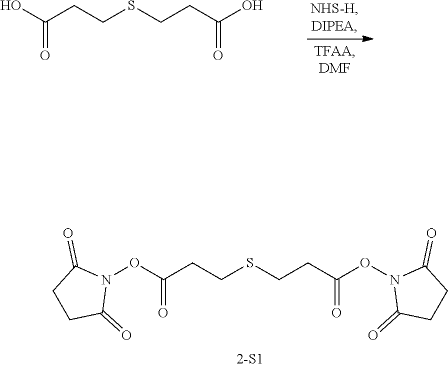 [SMILES: O=C(O)CCSCCC(=O)O ; O=C(CCSCCC(=O)ON1C(=O)CCC1=O)ON1C(=O)CCC1=O]

Bis(2,5-dioxopyrrolidin-1-yl) 3,3′-thiodipropionate (2-S1): To a solution of 3,3′-thiodipropionic acid (5.00 g, 28.1 mmol, 1.0 equiv), N-hydroxysuccinimide (12.9 g, 112 mmol, 4.0 equiv), and N,N-diisopropylethylamine (39.1 mL, 224 mmol, 8.0 equiv) in DMF (140 mL) at 0° C. was dropwise added to trifluoroacetic anhydride (15.8 mL, 112 mmol, 4.0 equiv). The orange solution was stirred for 2 h at 0° C., and then partitioned between EtOAc and brine. The aqueous layer was extracted with EtOAc (2×). The combined organic layers were washed with brine (5×), dried over anhydrous Na₂SO₄, and concentrated in vacuo. The resulting residue was purified via flash chromatography (40% EtOAc in CH₂Cl₂) to obtain NHS ester 2-S1 (8.21 g, 79%). ¹H NMR (500 MHz, DMSO-d6): δ 3.01 (t, J=7.0 Hz, 4H), 2.87 (t, J=7.0 Hz, 4H), 2.82 (s, 8H); ¹³C NMR (126 MHz, DMSO-d6): δ 169.9, 167.6, 31.4, 25.6, 25.4.

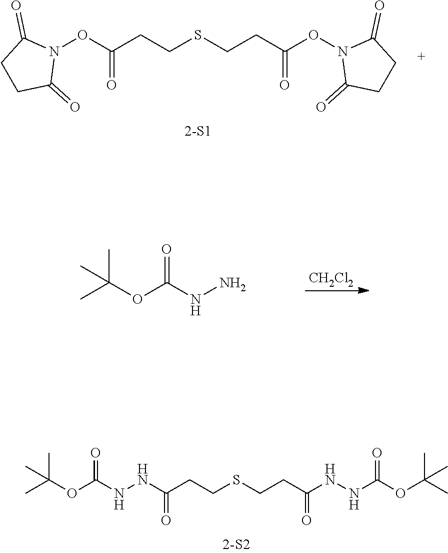

Di-tert-butyl 2,2′-(3,3′-thiobis(propanoyl))bis(hydrazine-1-carboxylate) (2-S2): To a slurry of NHS ester 2-S1 (2.73 g, 7.33 mmol, 1.0 equiv) in CH₂Cl₂(38 mL) was added t-butyl carbazate (1.94 g, 14.7 mmol, 2.0 equiv) to obtain a clear orange solution. After stirring for 15.5 h at rt, the slurry was vacuum filtered to obtain a white flakey precipitate. The precipitate was washed with additional CH₂₂ and then dried further in vacuo to obtain sulfide 2-S2 as a flakey white solid (2.28 g, 77%). Melting point: 187-190° C.; ¹H NMR (500 MHz, DMSO-d6): δ 9.56 (s, 2H), 8.71 (s, 2H), 2.69 (t, J=7.4 Hz, 4H), 2.352 (t, J=7.0 Hz, 4H), 1.39 (s, 18H); ¹³C NMR (125 MHz, DMSO-d6): δ 170.1, 155.2, 79.0, 33.8, 28.1, 26.6; HRMS (ESI-TOF) m/z calculated for C₁₆H₃₀N₄O₆SNa (M+Na)⁺ 429.1779, found 429.1760.

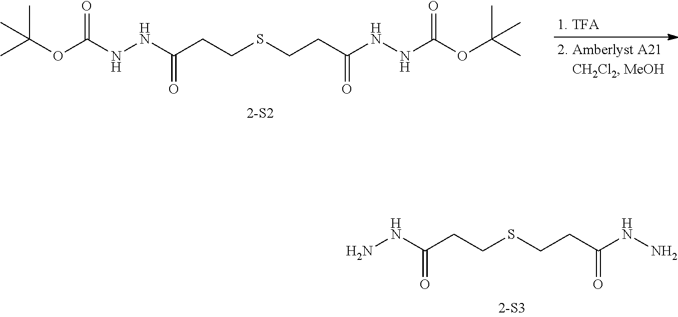 [SMILES: CC(C)(C)OC(=O)NNC(=O)CCSCCC(=O)NNC(=O)OC(C)(C)C ; NNC(=O)CCSCCC(=O)NN]

3,3′-Thiodi(propanehydrazide) (2-S3): Five separate vials each containing a solution of sulfide 2-S2 (200 mg, 0.492 mmol, 1.0 equiv) in trifluoroacetic acid (1.0 mL) was stirred at rt for 16 h and then concentrated in vacuo. The resulting residue from each vial was dissolved in a 1:1 solution of MeOH:CH₂Cl₂ and added to a mixture of Amberlyst A21 resin (4.0 g, 10.0 equiv by mass of salt product) in CH₂Cl₂. The mixture was stirred for 45 min at rt, after which the resin was filtered and washed with a 1:1 solution of MeOH:CH₂Cl₂. The filtrates were combined and concentrated in vacuo to obtain hydrazide 2-S3 as a tan solid (293 mg, 58%). ¹H NMR (600 MHz, DMSO-d6): δ 9.00 (s, 2H), 4.34 (s, br, 4H), 2.67 (t, J=7.3 Hz, 4H), 2.28 (t, J=7.3 Hz, 4H); ¹³C NMR (151 MHz, DMSO-d6): δ 169.9, 33.8, 26.9; HRMS (ESI-TOF) m/z calculated for C₆H₁₅N₄O₆S (M+H)⁺ 207.0911, found 207.0908.

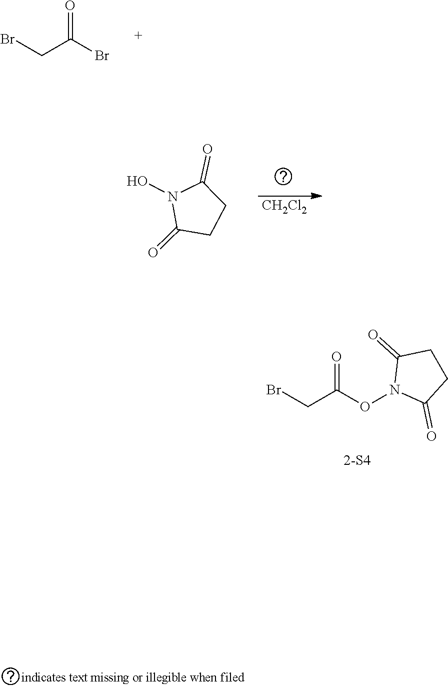

2,5-Dioxopyrrolidin-1-yl 2-bromoacetate (2-S4): To a solution of N-hydroxysuccinimide (1.32 g, 11.5 mmol, 1.0 equiv) in CH₂Cl₂ (15.0 mL) was dropwise added NEt₃ (1.92 mL, 13.8 mmol, 1.2 equiv) and a solution of bromoacetyl bromide (1.0 mL, 11.5 mmol, 1.0 equiv) in CH₂Cl₂ (15.0 mL) at 0° C. After stirring at 0° C. for 1 h, the reaction was quenched with saturated NaHCO₃ solution. The organic phase was washed with a saturated NaHCO₃ solution (2×), 1 M HCl (3×), and brine, dried over Na₂SO₄, and concentrated in vacuo to obtain NHS ester 2-S4 as a tan solid (1.95 g, 72%). ¹H NMR (500 MHz, CDCl₃): δ 4.10 (s, 2H), 2.86 (s, 4H); ¹³C NMR (126 MHz, CDCl₃) δ 168.6, 163.1, 25.7, 21.3.

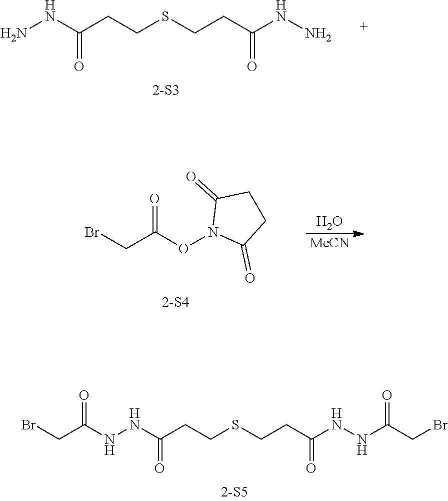 [SMILES: "MeCN" ; NNC(=O)CCSCCC(=O)NN ; O=C(CBr)ON1C(=O)CCC1=O ; O ; O=C(CBr)NNC(=O)CCSCCC(=O)NNC(=O)CBr]

3,3′-Thiobis(N′-(2-bromoacetyl)propanehydrazide) (2-S5): To a solution of hydrazide 2-S3 (293 mg, 1.42 mmol, 1.0 equiv) in H₂O (1.0 mL) was added a slurry of NHS ester 2-S4 (671 mg, 2.84 mmol, 2.0 equiv) in MeCN (1.0 mL). The vial containing ester 1-25 was washed with a MeCN:H₂O solution (1 mL) and the slurry was added to the reaction vial. After stirring at rt for 25 min the precipitate was vacuum filtered and washed with a 1:1 solution of MeCN:H₂O to obtain bromoacetamide 2-S5 as an off white powder (333 mg, 52%). Melting point: 166-170° C.; ¹H NMR (500 MHz, DMSO-d6): δ 10.38 (s, 2H), 10.12 (s, 2H), 3.91 (s, 4H), 2.72 (t, J=7.3 Hz, 4H), 2.43 (t, J=7.2 Hz, 4H); ¹³C NMR (126 MHz, DMSO-d6): δ 168.9, 164.5, 33.6, 27.1, 26.6; HRMS (ESI-TOF) m/z calculated for C₁₀H₁₆Br₂N₄O₄SNa (M+Na)⁺ 468.9152, found 468.9170.

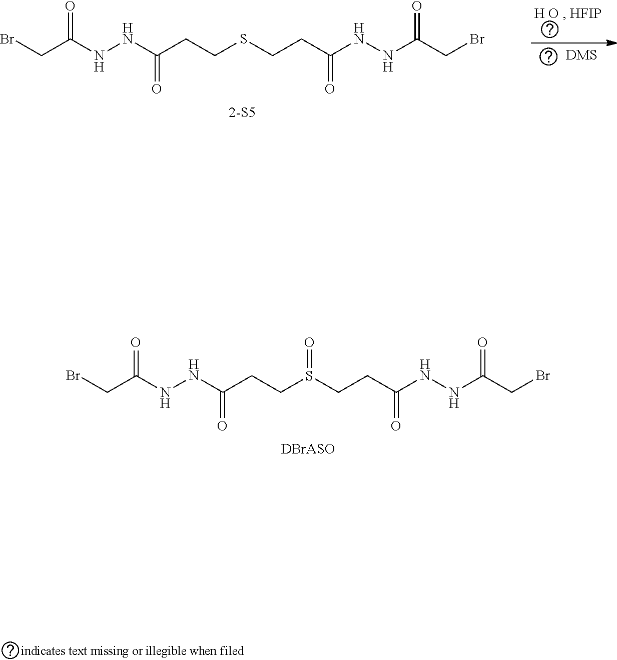 [SMILES: O=C(CBr)NNC(=O)CCSCCC(=O)NNC(=O)CBr ; O=C(CBr)NNC(=O)CCS(=O)CCC(=O)NNC(=O)CBr]

3,3′-Sulfinylbis(N′-(2-bromoacetyl)propanehydrazide) (DBrASO): To a solution of sulfide 2-S5 (400 mg, 0.893 mmol, 1.0 equiv) in HFIP (4.5 mL) at rt was added 30% aqueous H₂O₂ (0.18 mL, 1.78 mmol, 2.0 equiv). The tan slurry became clear and after stirring for 25 min at rt the reaction was slowly quenched with DMS (0.20 mL, 2.70 mmol, 3.0 equiv). A white solid precipitated out of solution as the DMS was added, and the slurry was stirred for an additional 10 min. The reaction mixture was vacuum filtered and the white precipitate was further dried in vacuo to obtain DBrASO as a white powder (235 mg, 57%). Melting point: 175-178° C.; ¹H NMR (600 MHz, DMSO-d6): δ 10.38 (s, 2H), 10.22 (s, 2H), 3.92 (s, 4H), 3.09-3.02 (m, 2H), 2.86-2.79 (m, 2H), 2.58 (t, J=6.8 Hz, 4H); ¹³C NMR (151 MHz, DMSO-d6): δ 168.6, 164.7, 46.1, 27.0, 25.8; HRMS (ESI-TOF) m/z calculated for C₁₀H₁₆Br₂N₄O₅SNa (M+Na)⁺ 484.9101, found 484.9118.

DBrASO Cross-linking of Synthetic Peptide. Synthetic peptide Ac-LR9 was dissolved in DMSO to a final concentration of 1 mM. DBrASO was added at 1:1 molar ratio to the peptide. The reaction was carried out at room temperature for one hour in the dark. The resulting samples were diluted to 10 pmol/μL in 3% ACN/2% formic acid prior to LC-MSⁿ analysis.

DBrASO Cross-linking of Bovine Serum Albumin and Cell lysates. 50 μL of 50 μM BSA in PBS buffer (2 mM TCEP, pH 7.6) was reacted with DBrASO at room temperature for 1 h in the dark. The HEK 293 cells were cultured in Dulbecco's modified Eagle's medium (DMEM) supplemented with 10% fetal bovine serum (FBS) and 1% penicillin-streptomycin until 90% confluence. After washing with PBS for three times, cell pellets were resuspended in lysing buffer (100 mM sodium chloride, 50 mM sodium phosphate, 10% glycerol, 2 mM ATP, 1 mM TCEP, 10 mM MgCl₂, 1×protease inhibiter (Roche), 1× phosphatase inhibitor, and 0.5% NP-40, pH 7.6). The cell debris were removed by centrifugation at 13,000 rpm for 15 min at 4° C. The supernatant was collected and adjusted to 1 mg/mL, followed by cross-linking with 10 mM DBrASO at room temperature for 1 h in dark.

Cross-linked Protein digestion. After cross-linking, the samples were transferred onto a 30 KDa centrifugal filter and washed with 8M urea to remove excess linker. The denatured proteins were reduced with 2 mM TCEP for 30 min and alkylated with 10 mM iodoacetamide for 30 min in the dark. After washing with 25 mM ammonium bicarbonate (ABC), the sample was resuspended in 8M urea/25 mM ABC buffer, and Lys-C (enzyme to protein ratio of 1:100) was added to the solution. The resulting mixture was incubated at 37° C. for 4 h. The concentration of urea was then reduced to 1.5 M for trypsin digestion (enzyme to protein ratio of 1:50) at 37° C. overnight. The digested peptides were acidified by adding 1% TFA to final concentration of 0.1% and desalted by Sep-Pak C18 cartridge prior to LC-MSⁿ analysis.

SEC-HpHt Fractionation of Cross-linked Peptides. Cross-linked peptides of cell lysates were further separated by peptide size exclusion chromatography coupled with high pH reverse phase tip fractionation (SEC-HpHt) as described in Jiao et al. (Analytical Chemistry 94:4236-4242 (2022)). Briefly, 250 μg of desalted peptides was dissolved in 30% ACN/0.1% FA, then loaded onto a Superdex™ Peptide 3.2/300 SEC column for separation. The two SEC fractions containing cross-linked peptides were collected, dried, dissolved in 160 μL of ammonia water (pH 10) and subjected to HpHt fractionation separately.

For HpHt separation, a pipette tip (200 μL) was first blocked with a layer of C8 membrane (Empore 3M). After filling with 5 mg of C18 solid phase (3 m, Durashell, Phenomenex). the tip was balanced with 90 μL of methanol, 90 μL of ACN and 90 μL of ammonia water (pH 10) sequentially. Then, dissolved peptides were loaded onto the tip and centrifuged at 1,200 rpm until the liquid level was close to the beads. After washing with another 90 μL of ammonia water (pH 10) for desalting, the peptides were eluted with increasing percentage of ACN in ammonia water (6%, 9%, 12%, 15%, 18%, 21%, 25%, 30%, 35%, and 50%). The 25%, 30%, 35% and 50% fractions were combined to 6%, 9%, 12% and 21% fractions, respectively. The final 6 fractions were vacuum dried and stored at −80° C. before LC-MSⁿ analysis.

LC-MSⁿ analysis. Cross-linked peptides were analyzed by LC-MSⁿ using an UltiMate 3000 RSLC coupled with an Orbitrap Fusion Lumos mass spectrometer. Samples were loaded onto a 50 cm×75 m Acclaim PepMap C18 column and separated over a 120 min gradient of 4% to 25% acetonitrile at a flow rate of 300 nL/min. The top 4 data-dependent MS³ acquisition method was used for the identification of DBrASO cross-linked peptides. Ions with charge of 4+ to 8+ in the MS¹ scan were selected for MS² analysis. The top 4 most abundant fragment ions in MS² scan were further fragmented by CID with a collision energy of 35%.

Identification of Cross-linked Peptides. Raw data were converted to mgf files by MSConvert (Protein Wizard 3.0.21288). Extracted MS³ spectra was subjected to Protein Prospector (v.6.3.3) for database searching (using Batch-Tag against SwissProt.2019.04. 08 random concatenated database). The mass tolerances were set as ±20 ppm for parent ions and 0.6 Da for fragment ions. Trypsin was set as the enzyme with three maximum missed cleavages allowed. A maximum of four variable modifications were also allowed, including cysteine carbamidomethylation, methionine oxidation, N-terminal acetylation, and N-terminal conversion of glutamine to pyroglutamic acid. Three defined DBrASO cross-linked modification on cysteine, including alkene (C₅H₆O₂N₂, +126 Da), thiol (C₅H₆O₂N₂S, +158 Da) and sulfenic acid (C₅H₈O₃N₂S, +176 Da) were also added as variable modifications. MSⁿ data were integrated via in-house software xl-Tools to identify cross-linked peptide pairs.

PPI Analysis and Structural Mapping. The PPI network of HEK293 cell lysates was derived based on DBrASO XL-MS data. BioPlex (bioplex.hms.harvard.edu/), BioGrid (thebiogrid.org/) and CORUM (mips.helmholtz-muenchen.de/corum) were used to determine protein complexes. Gene Ontology enrichment was performed using the R package “ClusterProfiler”. Cross-links mapping to high-resolution structures of protein complexes was performed as described in Wheat et al. (Proceedings of the National Academy of Sciences 118 (2021).

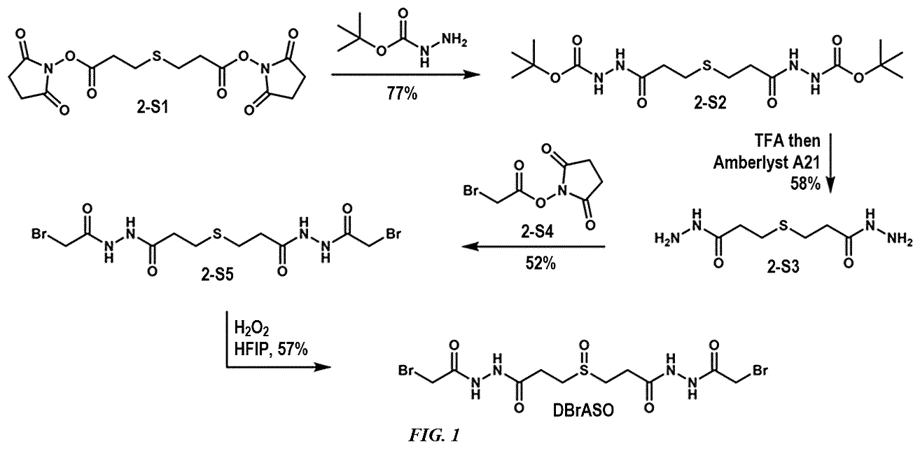

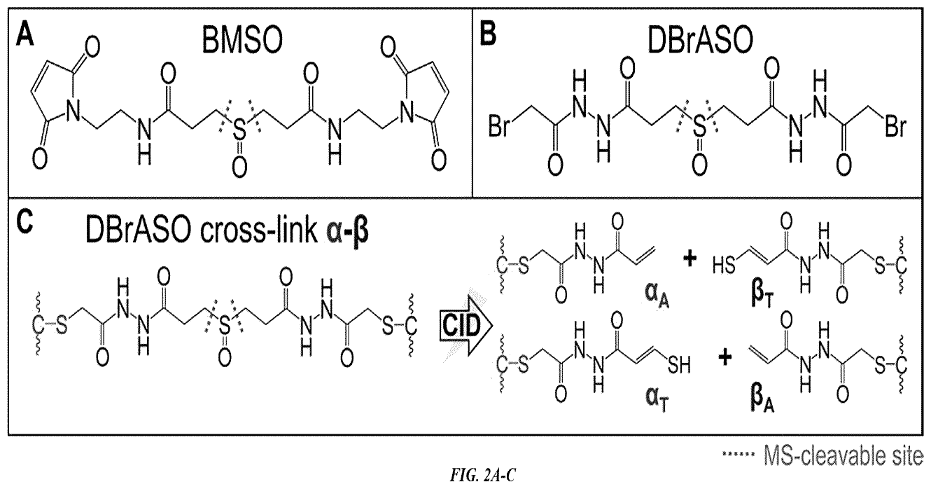

Design and Synthesis of a Novel MS-Cleavable Homobifunctional Cysteine Cross-Linker. To improve the specificity and homogeneity of cysteine cross-linking, an alternative sulfhydryl chemistry, i.e., the haloacetamide group was utilized to create a novel sulfoxide-containing MS-cleavable cysteine-reactive homobifunctional cross-linker (see FIG. 2A). Haloacetamides such as iodoacetamide and chloroacetamide have been used for cysteine alkylation prior to enzymatic digestion in proteomics studies. In this work, bromoacetamide was selected as the reactive group due to its comparable reactivity to iodoacetamide and ease of handling during synthesis. Thus, a homobifunctional cysteine crosslinker composed of two bromoacetamide groups connected by a spacer arm containing a central sulfoxide group adjacent to symmetrical MS-cleavable C—S bonds, DBrASO, was investigated (see FIG. 2B). The design of DBrASO was built upon the DHSO cross-linker, and the synthesis incorporated a late-stage sulfide to sulfoxide oxidation to minimize undesired reactivity (see FIG. 1). DBrASO has a spacer arm length of 16.4 Å, which is considerably shorter than BMSO (24.2 Å) and well within the distance range suited for mapping PPIs.

MS Characterization of DBrASO Cross-Linked Peptides. While DBrASO cross-linking is expected to result in different types of cross-linked peptides, the focus was on the characterization of DBrASO interlinked peptides due to their importance in delineating protein interactions and structures. Like other residue-specific sulfoxide-containing crosslinkers, such as DSSO and BMSO, cleavage of either MS-cleavable C—S bond physically separates the two crosslinked peptide constituents, yielding peptide fragment ion pairs (i.e., α_{A}/β_{S} or α_{S}/β_{A}). These resulting peptide fragments are modified with alkene (A) or sulfenic acid (S) moieties, remnants of DBrASO following collision-induced dissociation (CID). The sulfenic moiety typically undergoes dehydration to become a more stable and dominant unsaturated thiol (T) moiety. Therefore, the two dominant fragment pairs for a DBrASO cross-linked peptide α-β are expected to be α_{A}/β_{T} and α_{T}/β_{A} (see FIG. 2C), which are then subjected to MS³ analysis for unambiguous identification of cross-linked peptides.

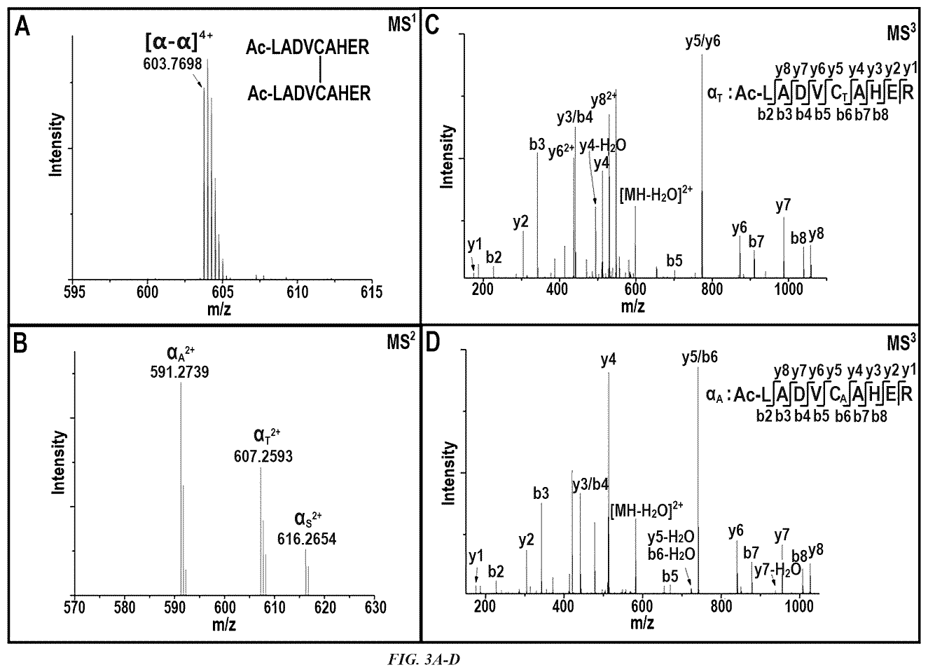

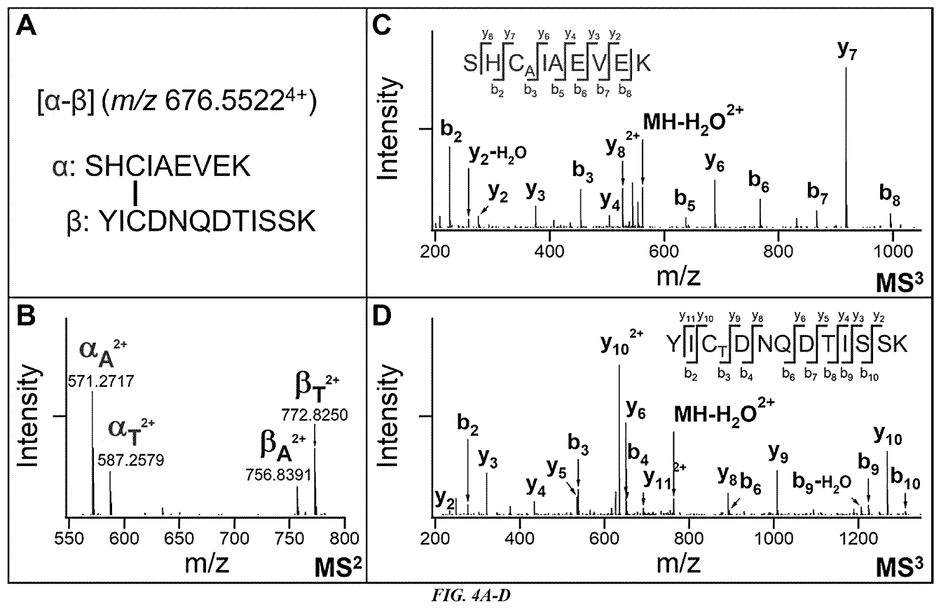

The fragmentation characteristics of DBrASO cross-linked peptides were first evaluated using a cysteine-containing synthetic peptide Ac-LR9 (i.e., Ac-LADVCAHER). MS² analysis of the DBrASO cross-linked Ac-LR9 homodimer (m/z 603.7698⁴⁺) resulted in three dominant fragments: αA (m/z 591.2379²⁺), α_{T} (m/z 607.2593²⁺), and αS (m/z 616.2654²⁺), with the alkene- and thiol-modified peptides being most dominant as anticipated (see FIG. 3). Subsequent MS³ analyses of α_{A} and α_{T} determined the C-C cross-link in the Ac-LR9 homodimer. These results demonstrate that CID-induced fragmentation of the DBrASO cross-linked peptide is like other sulfoxide-containing MS-cleavable cross-linked peptides.

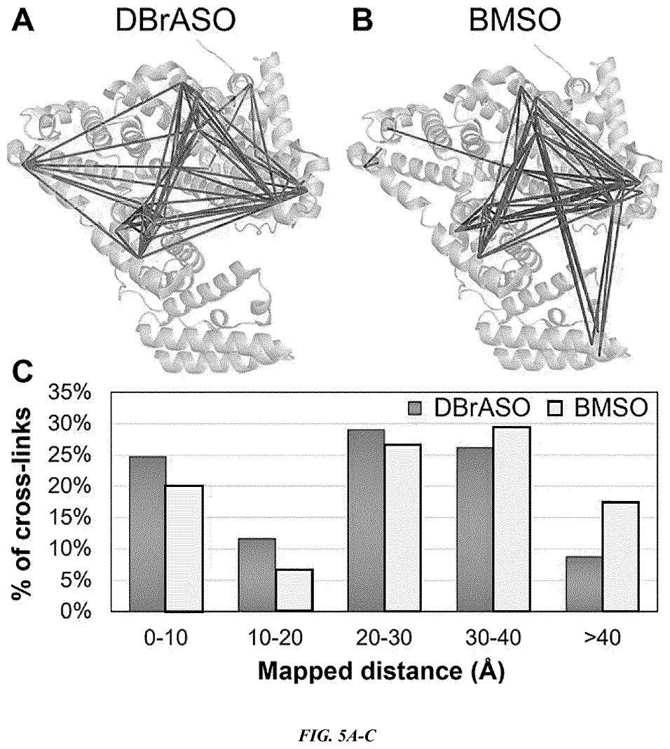

DBrASO XL-MS Analysis of BSA. To assess the efficiency of DBrASO for protein cross-linking, the readily available protein bovine serum albumin (BSA) was crosslinked due to its high cysteine content and common use in XL-MS studies. To characterize DBrASO cross-linking of BSA, LC-MSⁿ analysis was employed to identify DBrASO cross-linked peptides. As an example, MSⁿ analysis of a representative DBrASO interlinked peptide α-β of BSA (m/z 676.5522⁴⁺) is illustrated in FIG. 4. Fragmentation of the cross-link during MS² produced two major ion pairs, i.e., α_{A}/β_{T} and α_{T}/β_{A}, further confirming the expected fragmentation characteristics of DBrASO cross-linked peptides. MS³ sequencing of α_{A} (m/z 571.2717²⁺) and f_{T} (m/z 772.8250²⁺) determined their peptide sequences as ²⁸⁶SHCAIAEVEK²⁹⁴ (SEQ ID NO:2) and ³¹¹YICTDNQDTISSK³²² (SEQ ID NO:3) respectively. Together, the integration of MS¹, MS², and MS³ spectral data unambiguously identified a cross-link between C288 and C313 of BSA. In total, 69 unique C-C linkages were identified from three biological replicates (Table 1). 59.4% of the unique C-C linkages were found in all three replicates, displaying similar reproducibility to other sulfoxide-containing cross-linkers. To evaluate the interaction coverage observed by DBrASO, identified C-C linkages were plotted on a published BSA crystal structure (PDB: 4F5S) (see FIG. 5A). In total, 27 out of 35 cysteines were found to be cross-linked, indicating a high cysteine coverage. Based on the length of the DBrASO space arm (16.4 Å) and cysteine side chains (2.8 Å), as well as considering protein flexibility and dynamics, the maximum Cα-Cα distance between two DBrASO cross-linkable cysteine residues would be estimated to be ≤40 Å. Out of the 69 C-C linkages, 91.3% of them were considered satisfactory as they are below the max distance threshold (see FIG. 5B), supporting the validity of the identified cross-links.

TABLE 1
BMSO and DBrASO Inter-linked BSA residues and distances

| Residue A | Residue B | Distance Å | BMSO | DBrASO |
|---|---|---|---|---|
| C58 | C191 | 30.701 | | Yes |
| C58 | C192 | 29.749 | | Yes |
| C58 | C200 | 32.166 | | Yes |
| C86 | C125 | 23.640 | Yes | |
| C86 | C200 | 47.400 | Yes | |
| C86 | C312 | 35.417 | Yes | |
| C86 | C99 | 18.343 | Yes | |
| C99 | C147 | 36.394 | Yes | |
| C99 | C191 | 41.653 | | Yes |
| C99 | C200 | 43.896 | | Yes |
| C125 | C223 | 16.501 | Yes | |
| C147 | C200 | 11.452 | Yes | Yes |
| C147 | C223 | 35.403 | Yes | Yes |
| C147 | C288 | 36.989 | Yes | |
| C147 | C312 | 28.096 | Yes | |
| C147 | C415 | 40.621 | Yes | |
| C147 | C471 | 36.578 | Yes | |
| C147 | C484 | 38.611 | Yes | |
| C191 | C200 | 5.756 | Yes | Yes |
| C191 | C223 | 35.681 | | Yes |
| C191 | C301 | 25.682 | | Yes |
| C191 | C302 | 27.924 | | Yes |
| C191 | C312 | 23.452 | | Yes |
| C191 | C415 | 37.369 | | Yes |
| C191 | C471 | 32.840 | | Yes |
| C191 | C484 | 39.506 | | Yes |
| C191 | C510 | 46.549 | | Yes |
| C191 | C590 | 44.622 | | Yes |
| C192 | C200 | 8.321 | Yes | Yes |
| C192 | C223 | 37.339 | | Yes |
| C192 | C301 | 28.525 | | Yes |
| C192 | C302 | 30.494 | | Yes |
| C192 | C312 | 26.320 | | Yes |
| C192 | C471 | 36.034 | | Yes |
| C192 | C484 | 41.158 | | Yes |
| C192 | C510 | 48.922 | | Yes |
| C200 | C223 | 35.862 | Yes | Yes |
| C200 | C288 | 34.847 | Yes | Yes |
| C200 | C301 | 27.473 | Yes | |
| C200 | C302 | 30.238 | Yes | |
| C200 | C312 | 24.707 | Yes | Yes |
| C200 | C392 | 62.869 | Yes | |
| C200 | C415 | 33.208 | | Yes |
| C200 | C460 | 27.630 | Yes | |
| C200 | C461 | 29.099 | Yes | |
| C200 | C471 | 30.273 | | Yes |
| C200 | C484 | 37.979 | Yes | Yes |
| C200 | C510 | 44.161 | | Yes |
| C200 | C537 | 28.772 | | Yes |
| C200 | C590 | 40.494 | | Yes |
| C223 | C268 | 8.417 | Yes | |
| C223 | C269 | 5.259 | Yes | |
| C223 | C276 | 13.381 | Yes | |
| C223 | C288 | 26.379 | Yes | Yes |
| C223 | C301 | 26.271 | Yes | Yes |
| C223 | C302 | 27.395 | Yes | |
| C223 | C312 | 21.895 | Yes | Yes |
| C223 | C339 | 32.180 | | Yes |
| C223 | C392 | 36.103 | Yes | |
| C223 | C415 | 30.570 | | Yes |
| C223 | C460 | 25.112 | | Yes |
| C223 | C461 | 28.597 | | Yes |
| C223 | C484 | 12.687 | | Yes |
| C288 | C301 | 7.930 | Yes | |
| C288 | C302 | 5.203 | Yes | |
| C288 | C312 | 11.205 | Yes | Yes |
| C288 | C392 | 44.019 | Yes | |
| C288 | C415 | 40.502 | Yes | Yes |
| C288 | C484 | 37.129 | Yes | |
| C301 | C312 | 5.348 | Yes | Yes |
| C301 | C415 | 35.561 | Yes | |
| C301 | C460 | 27.269 | | Yes |
| C301 | C461 | 30.406 | | Yes |
| C301 | C581 | 56.402 | | Yes |
| C301 | C582 | 53.482 | | Yes |
| C302 | C312 | 8.270 | Yes | Yes |
| C302 | C415 | 38.956 | Yes | |
| C302 | C460 | 30.678 | | Yes |
| C302 | C461 | 33.878 | | Yes |
| C312 | C392 | 44.322 | Yes | |
| C312 | C415 | 31.513 | Yes | Yes |
| C312 | C460 | 23.166 | | Yes |
| C312 | C461 | 26.520 | | Yes |
| C312 | C471 | 20.567 | Yes | Yes |
| C312 | C484 | 30.371 | | Yes |
| C312 | C581 | 51.583 | | Yes |
| C312 | C582 | 48.751 | | Yes |
| C383 | C392 | 5.526 | Yes | Yes |
| C384 | C392 | 8.389 | Yes | Yes |
| C392 | C415 | 38.659 | Yes | |
| C392 | C471 | 33.488 | Yes | |
| C392 | C484 | 35.979 | Yes | |
| C415 | C460 | 8.358 | Yes | Yes |
| C415 | C461 | 5.520 | Yes | Yes |
| C415 | C471 | 12.255 | Yes | Yes |
| C415 | C484 | 24.612 | Yes | |
| C415 | C510 | 20.063 | Yes | |
| C460 | C471 | 5.688 | Yes | Yes |
| C460 | C484 | 22.489 | | Yes |
| C460 | C499 | 23.690 | Yes | |
| C460 | C500 | 23.752 | Yes | |
| C461 | C471 | 8.770 | Yes | Yes |
| C461 | C484 | 24.894 | | Yes |
| C461 | C499 | 25.457 | Yes | |
| C461 | C500 | 25.984 | Yes | |
| C471 | C484 | 20.608 | Yes | Yes |
| C471 | C499 | 21.175 | Yes | |
| C471 | C500 | 20.993 | Yes | |
| C471 | C510 | 18.369 | Yes | Yes |
| C484 | C499 | 8.491 | Yes | |
| C484 | C500 | 5.691 | Yes | |
| C484 | C510 | 12.514 | Yes | |
| C499 | C510 | 5.652 | | Yes |
| C499 | C500 | 3.846 | | Yes |
| C500 | C510 | 8.788 | Yes | Yes |
| C537 | C581 | 8.645 | | Yes |
| C537 | C582 | 5.846 | | Yes |

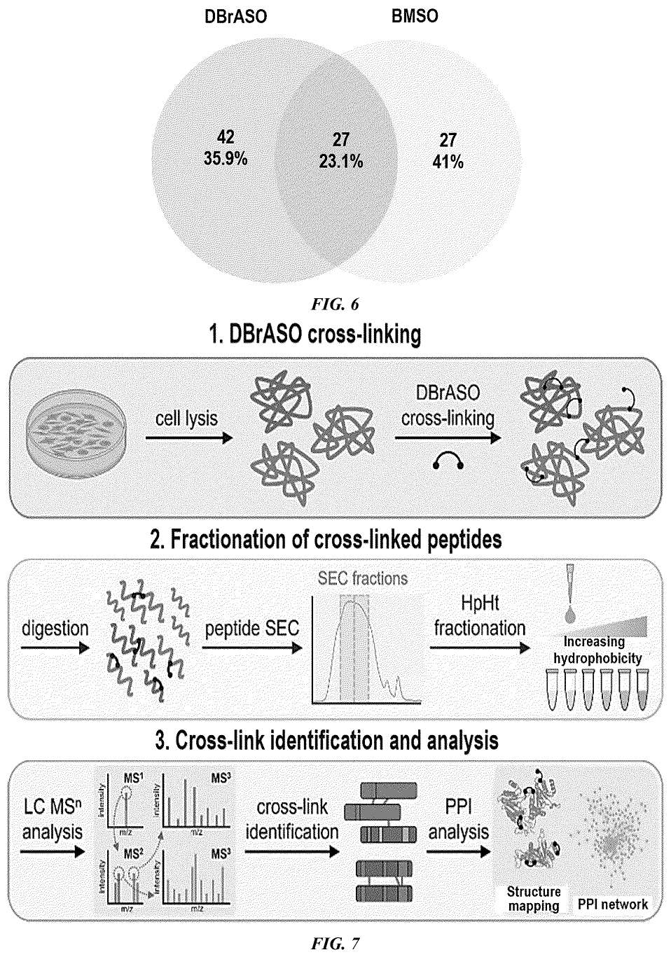

To examine whether the differences in cysteine-labeling efficiency between haloacetamide and maleimide functional groups could impact BSA cross-linking, BMSO cross-linking on BSA was performed using the same conditions as DBrASO. A total of 75 BMSO C-C linkages were identified from three biological replicates, and 92% of them were considered satisfactory (≤45 Å) (see FIG. 5B). Although the total number of unique C-C linkages identified for each linker was similar, they only shared 23.1% of identified linkages in common (see FIG. 6) significantly lower than the overlap among their respective biological replicates. The limited commonality suggests discernable differences between the efficiency of DBrASO and BMSO cross-linking. Although DBrASO (16.4 Å) is shorter than BMSO (24.2 Å), the average distances of their cross-links were 23.9±13.3 Å (24.6 Å median) for DBrASO and 26.9±13.6 Å (28.6 Å median) for BMSO. Out of the 42 C-C linkages that were uniquely identified by DBrASO, five were longer than 40 Å. For comparison, 12 out of the 48 BMSO unique cross-linked linkages were longer than 40 Å and six of them were longer than 45 Å. Therefore, the observed differences between DBrASO and BMSO cross-linking of BSA are most likely due to the differences in the two linkers including their chemical structures, reaction kinetics, tendency to hydrolyze, product heterogeneity, and accessibility to cross-linkable sites. In addition, owing to the structural differences in BMSO and DBrASO cross-links, there will be variability in the separation and detectability of DBrASO and BMSO cross-linked peptides during LC-MSn analysis. Taken together, the results demonstrate that DBrASO is effective for protein cross-linking and yields cross-links complementary to BMSO, thus increasing PPI coverages through C-C linkages.

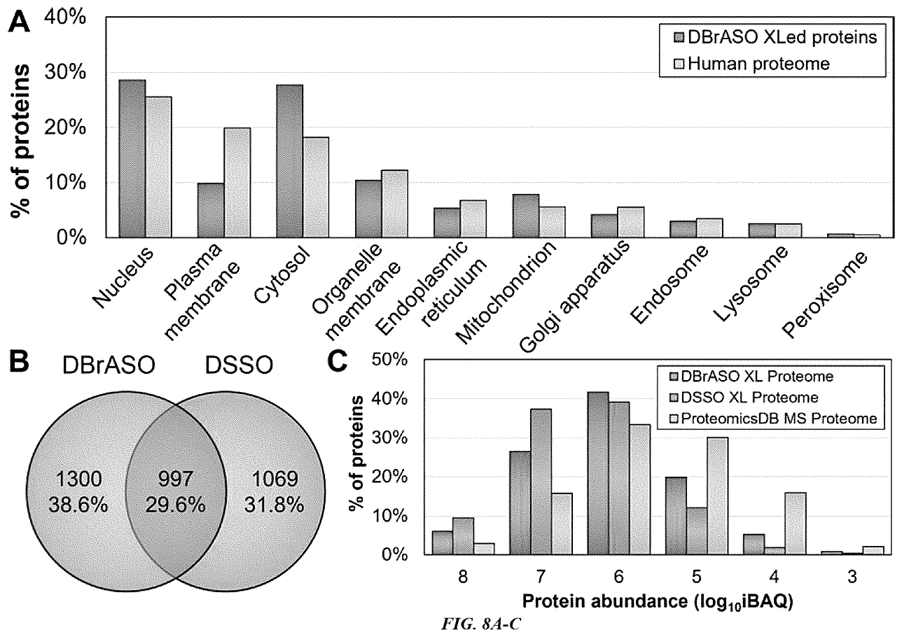

DBrASO XL-MS Analysis of HEK293 Cell Lysates. Recent advances in XL-MS technologies have successfully enabled proteome-wide XL-MS studies using lysine-reactive cross-linkers. However, other cross-linking chemistries which complement lysine-reactive reagents in proteome-wide PPI mapping have not yet been investigated. Previous XL-MS analyses of proteins and protein complexes have proven the benefits of using multiple cross-linking chemistries to expand PPI mapping and enhance the precision of structural modeling. To explore the feasibility of cysteine-reactive cross-linkers for proteome-wide XL-MS studies, DBrASO cross-linking of HEK293 cell lysates were performed (see FIG. 7). To enhance the detectability and identification of cross-linked peptides from complex samples, a recently developed two-dimensional fractionation method was employed by coupling peptide size-exclusion chromatography (SEC) with tip-based high-pH reverse phase (HpHt) separation (SEC-HpHt)15 to enrich DBrASO cross-linked peptides from cell lysate digests. This workflow resulted in a total of 12 SEC-HpHt fractions for each proteome-wide XLMS experiment. Here, we performed three biological replicates, yielding a total of 36 fractions for LC-MSⁿ analysis. As a result, 27,809 cross-link peptide spectrum matches (CSMs) were obtained, corresponding to 11,478 unique DBrASO crosslinked peptides that represent 8222 unique C-C linkages from 2297 human proteins. Here, the 8222 unique C-C linkages describe 1037 interprotein and 7185 intraprotein interactions.

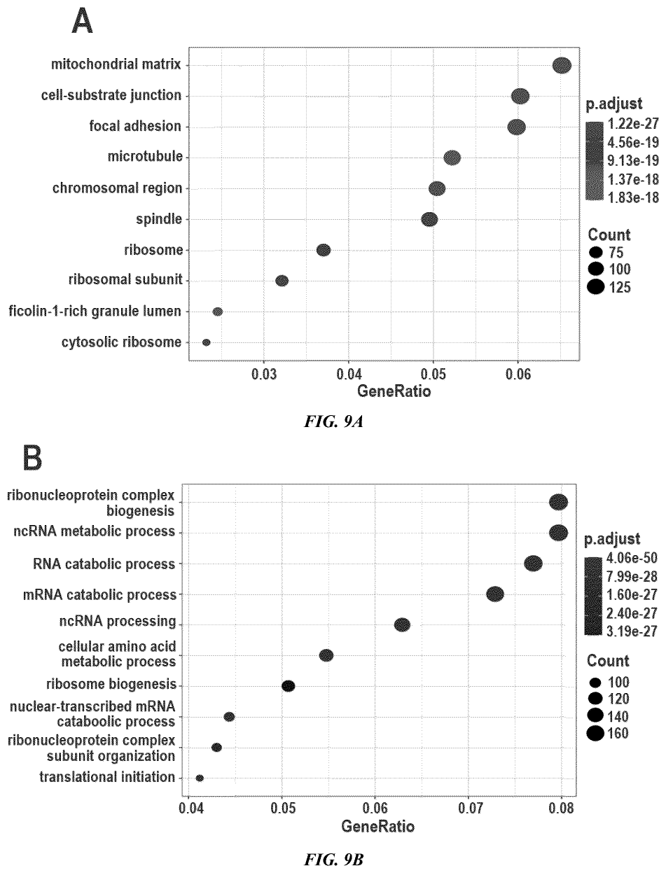

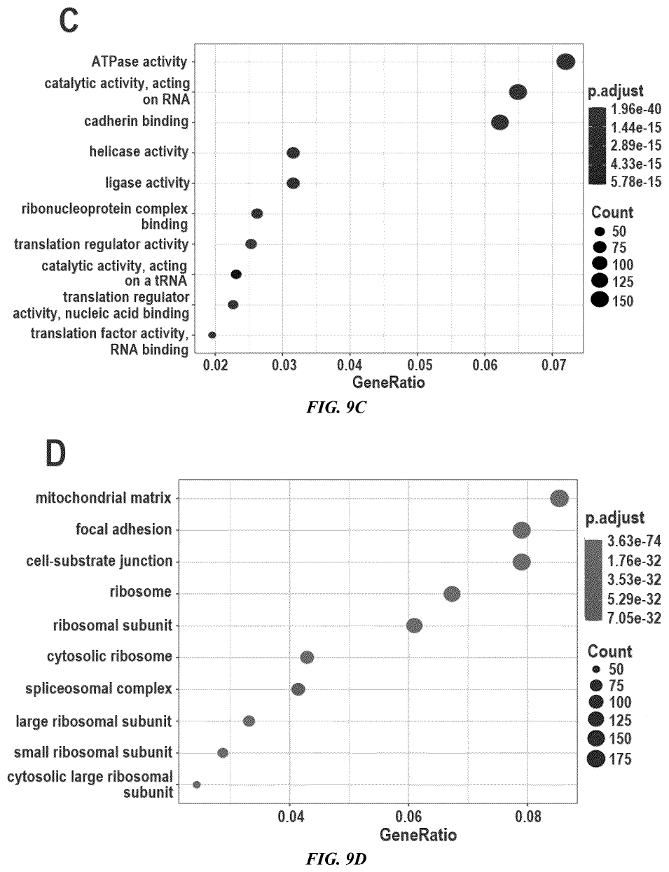

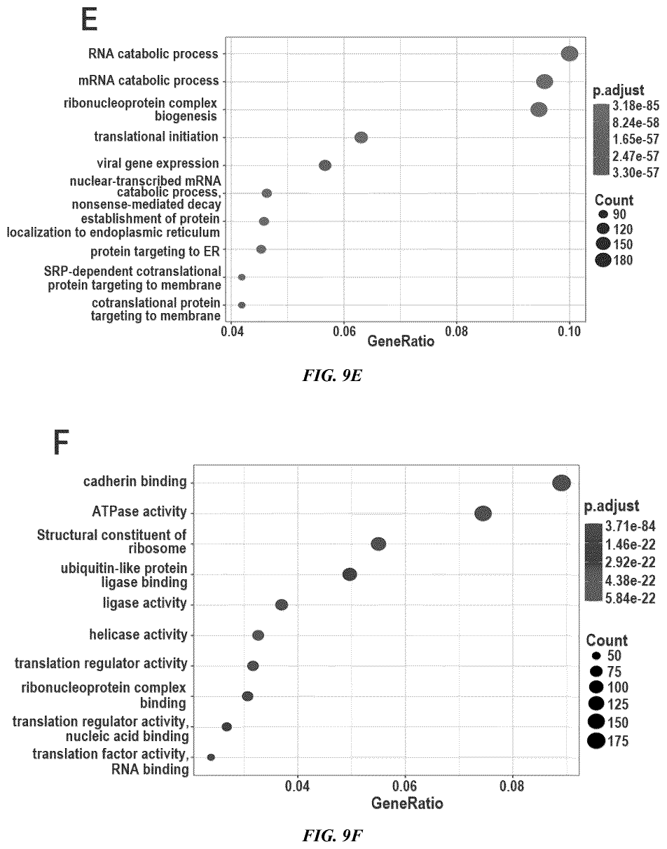

Comparisons of DBrASO and DSSO XL-Proteomes. To illustrate the differences between cysteine and lysine crosslinking chemistries, the identified 2297 DBrASO XL-MS proteome was compared with a previously published DSSO XL proteome of HEK293 cell lysates. Similar to DSSO, DBrASO cross-linked proteins were enriched in various subcellular compartments, including the nucleus, cytosol, and mitochondria (see FIG. 8A). Of the proteins identified by DBrASO crosslinking crosslinking, 997 were shared with the DSSO XL-proteome. The remaining 1300 proteins were unique to DBrASO, expanding the overall proteome coverage (see FIG. 8B). Gene Ontology (GO) analysis indicated that similar cellular components were found for both DBrASO and DSSO XL-proteomes, with an enrichment in the mitochondrial matrix, focal adhesion, cell substrate junction, and ribosomes. In addition, similar biological processes, such as RNA metabolic process and ribonucleoprotein complex biogenesis, and molecular functions, such as protein binding or kinase activity, were enriched in both XL-MS data sets (see FIG. 9).

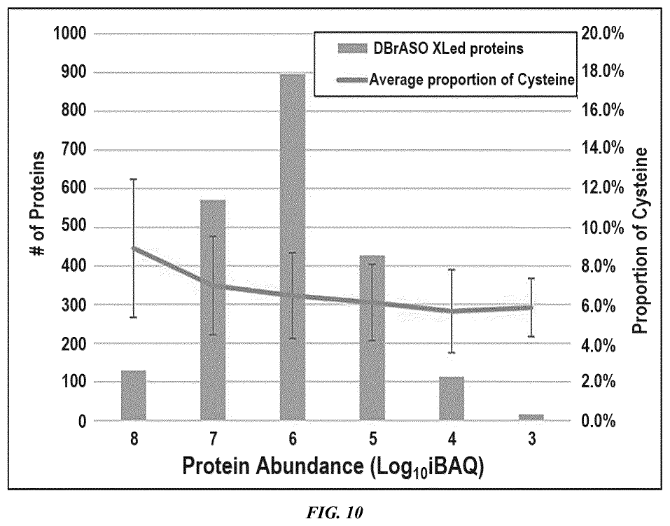

The abundance distribution of XL proteomes were plotted using the intensity-based absolute quantification (iBAQ) values of the HEK293 MS proteome in ProteomicsDB. 38 Out of the 2297 DBrASO-identified proteins, 2152 were correlated with iBAQ values, and their distribution indicated that high-abundance proteins were well-represented in the cysteine XL-proteome (see FIG. 8C), like lysine XLproteomes. This result was unexpected, as the yield of cross-linking data is substoichiometric and heavily dependent on protein abundance. Interestingly, DBrASO appeared to uncover slightly more low-abundance proteins (Log 10 iBAQ≤5) than DSSO15 (556 vs 275), suggesting that cysteine-reactive cross-linkers may be helpful in capturing certain lower abundance PPIs. To understand the relationship between cysteine occurrence and protein abundance, we plotted their distribution correlation (see FIG. 10). As shown, the average occurrence of cysteines did not correlate to protein abundance. Next, the total number of interprotein and intraprotein cross-links containing low-abundance proteins (Log 10 iBAQ≤5) was compared. Among the 891 unique C-C linkages involving the 556 DBrASO cross-linked low-abundance proteins, 84 represent interprotein and 807 represent intraprotein cross-links. In comparison, among the 591 unique K-K linkages from the 275 DSSO cross-linked proteins, 286 depict interprotein and 305 represent intraprotein cross-links. This indicates that most of the cross-links containing low abundance proteins from DBrASO XL data were obtained through intraprotein interactions. As the occurrence of lysines is significantly higher than that of cysteines in the proteome and on protein interaction interfaces, higher number of interprotein cross-links would be identified with DSSO than DBrASO. For the same reason, the total number of DSSO cross-links from high-abundance proteins would be much higher than that of DBrASO, leading to less identification of DSSO cross-links from low-abundance proteins. Therefore, the observed differences in DBrASO and DSSO XL-proteomes are likely attributed to multiple factors including cross-linking chemistry, the availability of cross-linkable residues, and the detectability of resulting cross-linked peptides. Nonetheless, the results suggest DBrASO can capture additional PPIs to help expand proteome coverage in XL-MS experiments.

Examination of Cross-Links by Structural Mapping. The DBrASO XL-proteome of HEK293 cell lysates is composed of 2297 proteins, in which 975 CORUM protein complexes were found and 74.5% of them were identified with more than 50% protein composition of each protein complex. Based on the number of C-C linkages, the most representative complexes are the cytoplasmic ribosome, CCT, 26S proteasome, and pre-mRNA spliceosome complexes. To assess the validity of DBrASO cross-links, a total of 1380 C-C linkages (879 intraprotein and 501 interprotein) were mapped onto available high-resolution structures of 126 protein complexes. While 717 out of the 879 intraprotein C-C linkages (81.6%) corresponded to Cα-Cα distances below the DBrASO threshold (≤40 Å), the majority of the 501 interprotein C-C linkages were found to be nonsatisfactory. However, it is noted that 97% of the violating interprotein cross-links were determined to be associated with ribosomal proteins (460/475). This is not surprising as the ribosomal complex is known to be highly dynamic, and various lysine-based XL-MS data sets of purified samples and cell lysates have revealed many nonsatisfactory interprotein cross-links from ribosomal proteins. Therefore, DBrASO XL data further support the structural plasticity of the ribosomal complex.

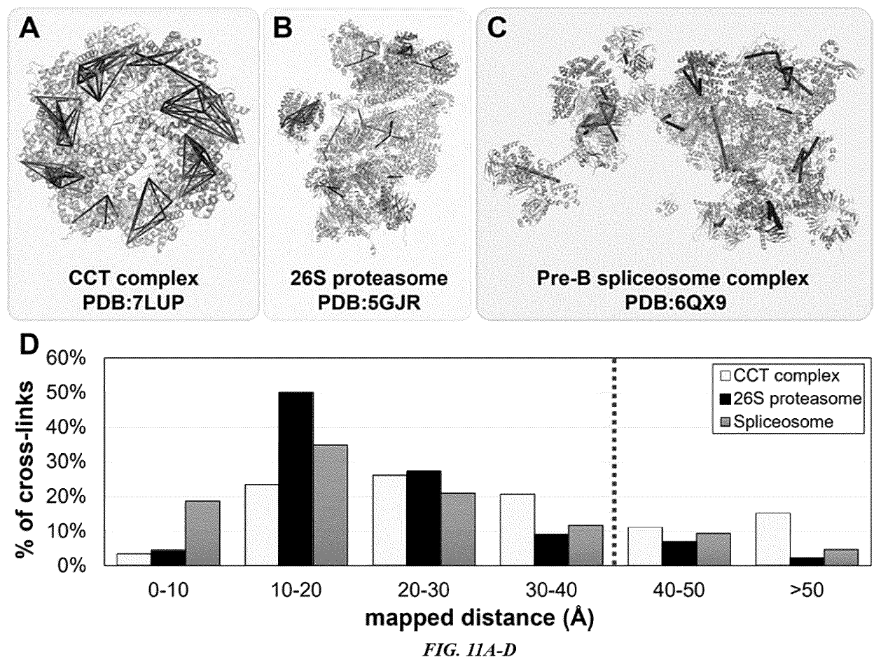

A well-represented complex in the data set is the eight subunit CCT complex, identified with 153 C-C cross-links describing 148 intra-subunit and five intersubunit interactions involving all eight subunits. Out of the 145 C-C linkages that were able to be mapped onto the CCT complex structure (PDB: 7LUP), the majority (74%) were satisfied with the Ca-cross-link satisfaction was determined to be 91% (40/44) and 86% (37/43), respectively (see FIG. 11B-D). Collectively, these results suggest that most DBrASO cross-links matched well with known protein structures, in line with results obtained using lysine-reactive cross-linkers.

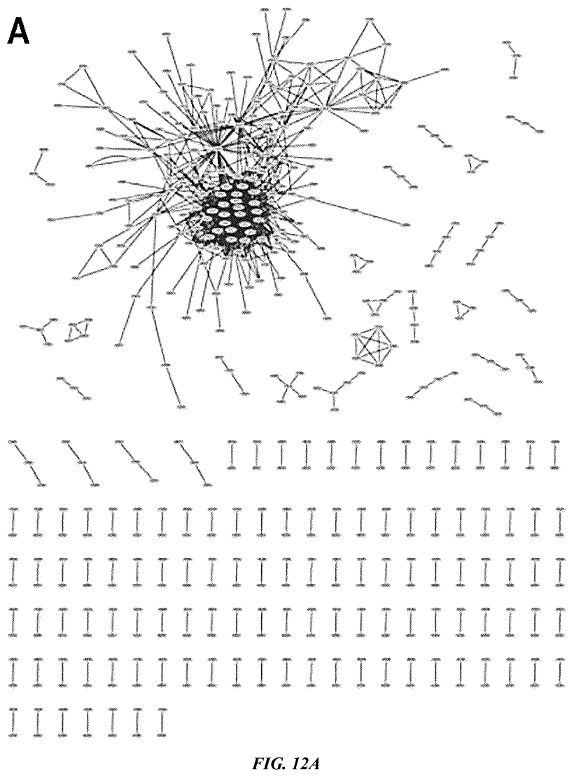

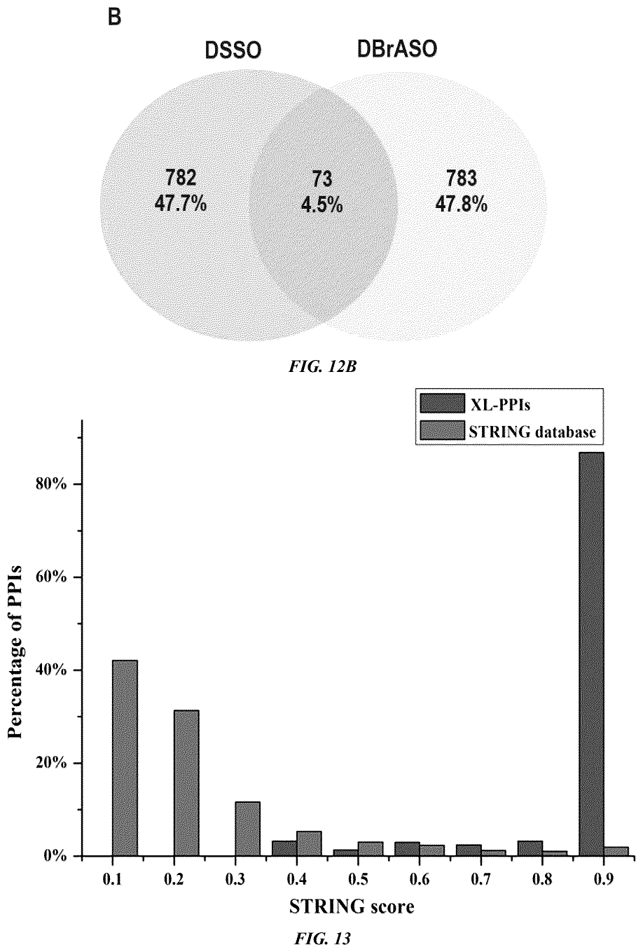

DBrASO XL-PPI Network of HEK293 Cell Lysates. To visualize the identified PPIs, an XL-PPI network comprising 505 nodes and 856 edges was generated, denoting 856 interprotein interactions (see FIG. 12A). Although the number of XL-PPIs captured by DBrASO and DSSO is similar (856 vs 855), their overlap is only 4.5% (see FIG. 12B), suggesting substantial differences in PPI mapping. For the 323 XL-PPIs that are present in the STRING database, more than 86% have a STRING score above 0.9 (see FIG. 13), which is like other proteome-wide XL-MS results, suggesting high confidence in the PPIs captured by DBrASO cross-linking. In comparison to current BioPlex and BioGrid databases as well as the published proteome-wide XL-MS data, 570 out of the identified 856 PPIs are known and 286 are novel.

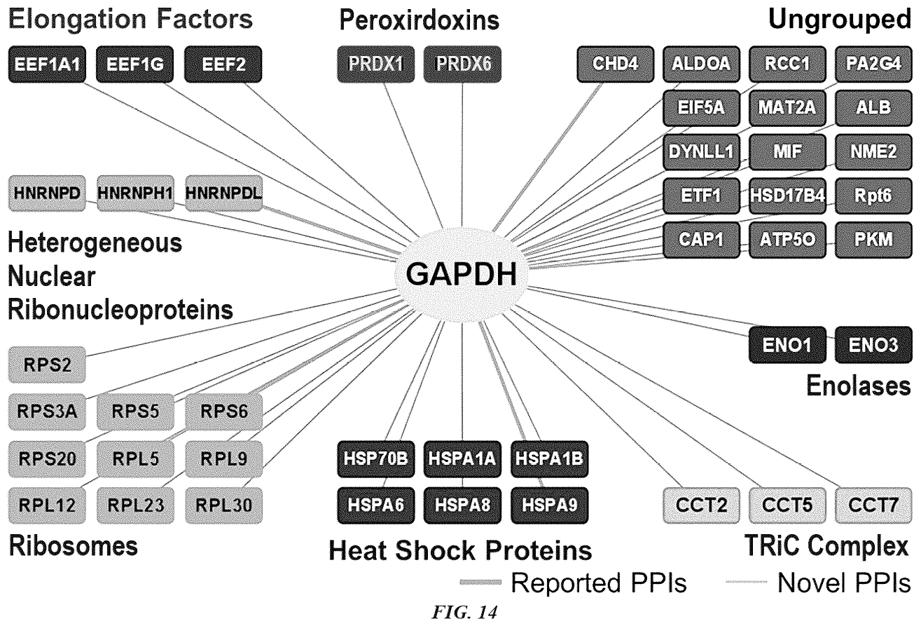

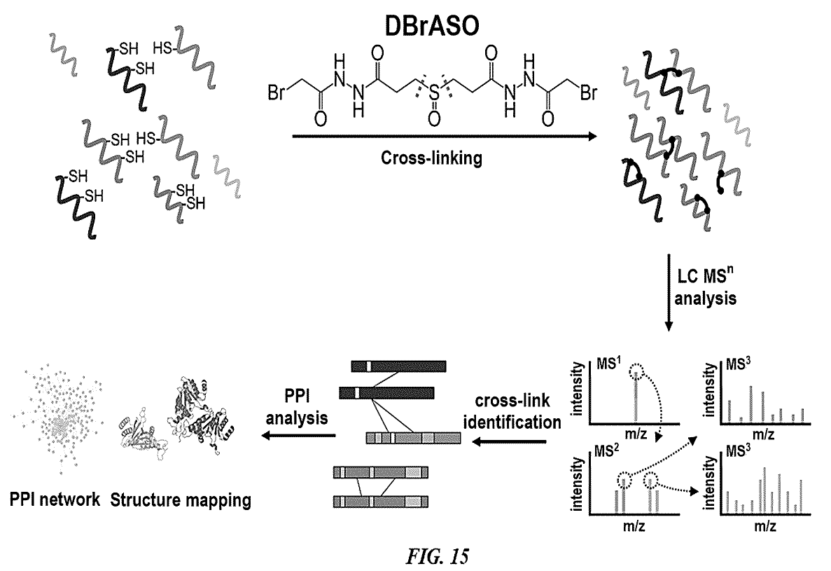

Among the novel PPIs, 128 correspond to interactions involving ribosomal proteins. For instance, multiple interaction contacts between UBA52 and 40S/60S ribosomal proteins were uniquely identified by DBrASO cross-linking but not by lysine-reactive reagents. This is due to the fact that the N-terminus of UBA52 (also called ubiquitin-60S ribosomal protein L40) has identical sequence to ubiquitin (1-76 AA). Thus, any cross-linked peptides involving the N-terminus of UBA52 (1-76) would be ambiguous as they could belong to ubiquitin and/or other ubiquitin precursors. While multiple lysines exist at the C-terminus (77-128 AA) of UBA52, crosslinked peptides involving the C-terminus have not been reported using lysine-reactive cross-linkers. Here, cysteine XL by DBrASO produced physical evidence to support the direct interaction of UBA52 with ribosomal proteins due to the identification of its unique C-terminal peptide (¹¹⁵CGHTNNLRPK¹²⁴) (SEQ ID NO:4). Additionally, 34 novel interactions identified here are related to GAPDH, including its interactions with heat shock proteins, 40S ribosomal, 60S ribosomal, TRiC-complex, and other proteins (see FIG. 14). All these interactions involve C247 of GAPDH, indicating that this region is in proximity to GAPDH interactors. Although GAPDH is a relatively abundant protein, its interactions with other proteins have rarely been described in previous DSSO XL-MS data sets of cell lysates. While enrichable lysine-reactive alkyne-A-DSBSO was able to capture several GAPDH-related PPIs, 14 only two PPIs were shared with those captured by DBrASO (GAPDH-HNRNPDL and GAPDH-RPL30). Interestingly, 29 out of the 65 interprotein DSBSO K-K linkages from GAPDH involve K251, K259, K260, and K263,14 which are very close to C247 in both sequence and structure, reaffirming GADPH interactions captured by DBrASO in HEK293 cell lysates. Moreover, an interaction between CCT2:C535 and TUBB:C12 was identified, which was not uncovered by previous lysine-reactive XL-MS studies. While it is known that CCT complex regulates the folding of tubulin, the details in their physical interactions have not been reported before. The proximity between the C-terminus of CCT2 and N-terminus of TUBB identified here correlates well with a recently published 3D structure of nanobody and the tubulin bound human TRiC complex (PDB: 7NVN) although the complete C-terminal structure of CCT2 was not fully resolved. Collectively, the results have demonstrated the complementarity of DBrASO cross-linking to lysine-reactive reagents in proteome-wide PPI profiling.

A number of embodiments have been described herein. Nevertheless, it will be understood that various modifications may be made without departing from the spirit and scope of this disclosure. Accordingly, other embodiments are within the scope of the following claims.

1. A mass spectrometry (MS)-cleavable haloacetamide-based cross-linker comprising:
one or more terminal haloacetamide groups;
a single, centrally located sulfoxide group; and
one or more MS-cleavable bonds;
wherein the MS-cleavable haloacetamide-based cross-linker is configured to specifically interact with cysteine groups for mapping intra-protein interactions in a protein, or inter-protein interactions in a protein complex, or combinations thereof.

2. The MS-cleavable haloacetamide-based cross-linker of claim 1, wherein the one or more MS-cleavable bonds is/are C—S bond(s) adjacent to the single, centrally located sulfoxide group.

3. The MS-cleavable haloacetamide-based cross-linker of claim 1, wherein the mass spectrometry (MS)-cleavable haloacetamide-based crosslinker has 2 terminal haloacetamide groups that are located equal distant to the single, centrally located sulfoxide group.

4. The MS-cleavable haloacetamide-based cross-linker of claim 1, wherein the haloacetamide group has a structure of: 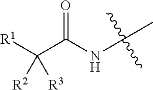
wherein, R¹-R³ are individually selected from H, D and halo, wherein at least one of R¹-R³ is a halo.

5. The MS-cleavable haloacetamide-based cross-linker of claim 4, wherein R¹ is Br; and R²-R³ are H.

6. The MS-cleavable haloacetamide-based cross-linker of claim 1, wherein the MS-cleavable haloacetamide-based cross-linker further comprises a first linker group, wherein one end of the first linker arm is connected to the single, centrally located sulfoxide group and the other end of the first linker group is connected to one of the terminal haloacetamide groups.

7. The MS-cleavable haloacetamide-based cross-linker of claim 6, wherein the MS-cleavable haloacetamide-based cross-linker further comprises a second linker group, wherein one end of the second linker group is connected to the central sulfoxide group and the other end of the second linker group is connected to another terminal haloacetamide group, and wherein the first linker group and the second linker groups comprise identical functional groups and are symmetric.

8. The MS-cleavable haloacetamide-based cross-linker of claim 1, wherein the MS-cleavable haloacetamide-based cross-linker is DBrASO having the structure of:

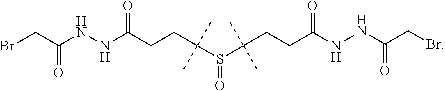

9. A mass spectrometry (MS)-cleavable haloacetamide-based cross-linker having the structure of Formula (I): 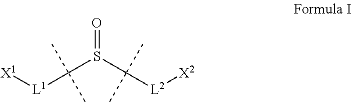
wherein,
X¹ and X² are independently selected haloacetamide groups;
L¹ and L² are independently selected linker groups; and
wherein the dashed line indicates a MS-cleavable bond.

10. The MS-cleavable haloacetamide-based cross-linker of claim 9, wherein X¹ and X² comprises the structure of 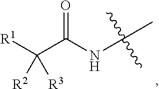
wherein R¹-R³ are individually selected from H, D or halo, and wherein at least one of R¹-R³ is a halo.

11. The MS-cleavable haloacetamide-based cross-linker of claim 10, wherein R¹ is Br; and R²-R³ are H.

12. The MS-cleavable haloacetamide-based cross-linker of claim 9, wherein L¹ and L² are individually selected from an optionally substituted (C₁-C₁₂)alkyl, an optionally substituted (C₁-C₁₂)alkenyl, 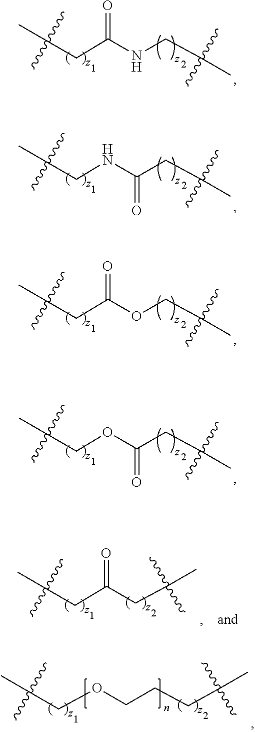
wherein, z¹ is an integer selected from 0, 1, 2, 3, 4, 5, 6, 7, and 8; z² is an integer selected from 1, 2, 3, 4, 5, 6, 7, and 8; and n is an integer selected from 1, 2, 3, and 4.

13. The MS-cleavable haloacetamide-based cross-linker of claim 12, wherein L¹ and L² are selected from, 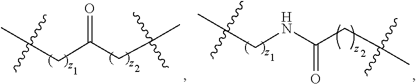
wherein, z¹ is an integer selected from 0, 1, 2, 3, and 4; and z² is an integer selected from 1, 2, 3, and 4.

14. The MS-cleavable haloacetamide-based cross-linker of claim 13, wherein L¹ and L² are 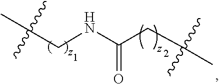
wherein, z¹ is an integer selected from 0, 1, 2 and 3; and z² is an integer selected from 2, 3, and 4.

15. The MS-cleavable haloacetamide-based cross-linker of claim 14, wherein L¹ and L² are selected from

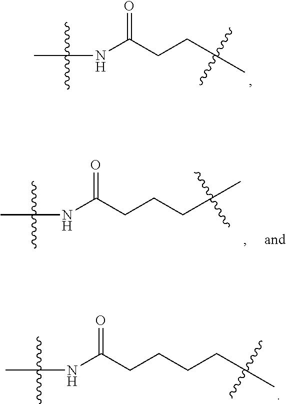 [SMILES: *CCC(=O)N* ; *CCCC(=O)N* ; *CCCCC(=O)N*]

16. The MS-cleavable haloacetamide-based cross-linker of claim 15, wherein the MS-cleavable haloacetamide-based cross-linker has a structure of: 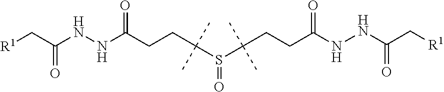
wherein, R¹ is a halo selected from Cl, Br, and I.

17. A method for mapping intra-protein interactions in a protein, inter-protein interactions in a protein complex, or any combination thereof, the method comprising:
contacting the protein and/or the protein complex comprising a plurality of cysteine moieties with the MS-cleavable haloacetamide-based cross-linker of claim 1 to form a cross-linked product;
digesting the cross-linked product to form a plurality of fragments, wherein a portion of the plurality of fragments comprises cross-linked peptide fragments; and
identifying and analyzing cross-linked peptide fragments using tandem mass spectrometry (MSⁿ) to map intra-protein interactions in the protein and/or inter-protein interactions in the protein complex.

18. The method of claim 17, wherein a data-dependent MS³ acquisition method is used for identifying and analyzing the cross-linked peptide fragments.

19. A method for mapping global protein-protein interactions (PPIs) from a sample comprising a plurality of proteins;
contacting the sample comprising a plurality of proteins with the MS-cleavable haloacetamide-based cross-linker of claim 1 to form crosslinked proteins;
digesting the crosslinked proteins to form crosslinked protein fragments or peptides;
isolating fractions that are enriched with cross-linked protein fragments or peptides in the sample;
analyzing the fractions using tandem mass spectrometry (MSⁿ) and protein database searching to identify cross-linked protein fragments or peptides; and
mapping the identified cross-linked protein fragments or peptides to generate a global structural map of PPIs.

20. The method of claim 19, wherein the fractions are isolated by using peptide size exclusion chromatography coupled with high pH reverse phase tip fractionation.


SEQUENCE LISTING (4 sequences)

SEQ ID NO: 1 - protein, 9 aa, synthetic construct
LADVCAHER

SEQ ID NO: 2 - protein, 10 aa, synthetic construct
SHCAIAEVEK

SEQ ID NO: 3 - protein, 13 aa, synthetic construct
YICTDNQDTISSK

SEQ ID NO: 4 - protein, 10 aa, synthetic construct
CGHTNNLRPK